(19) United States
(12) Patent Application Publication
(10) Pub. No.: US 2016/0075765 A1
(43) Pub. Date: Mar. 17, 2016

(54) MODIFIED COLLAGEN MOLECULES

(71) Applicant: AGENCY FOR SCIENCE, TECHNOLOGY AND RESEARCH, Singapore (SG)

(72) Inventors: Song-Gil Lee, Singapore (SG); Su Seong Lee, Singapore (SG); Jaehong Lim, Singapore (SG)

(21) Appl. No.: 14/890,037

(22) PCT Filed: May 9, 2014
(86) PCT No.: PCT/SG2014/000204

§ 371 Date: Nov. 9, 2015

(30) Foreign Application Priority Data
May 9, 2013 (SG) 201303597-7

(51) Int. Cl. C07K 14/78; A61Q 19/08; A61K 8/65

(52) U.S. Cl. CPC C07K 14/78; A61K 8/65; A61Q 19/08; A61K 38/00

(57) ABSTRACT

This invention relates to modified collagen molecules, in particular, modified collagen-glycosaminoglycan composites. This invention also provides methods for stabilizing collagen molecules and uses of these collagen molecules.

### CROSS-REFERENCE TO RELATED APPLICATIONS

This application claims the benefit of priority of Singapore application No. 201303597-7, filed May 9, 2013, the contents of it being hereby incorporated by reference in its entirety for all purposes.

### FIELD OF THE INVENTION

This invention is in the field of biomaterials and their synthesis, in particular, collagen molecules.

### BACKGROUND OF THE INVENTION

A biomaterial is a natural or synthetic material that is able to interact in a biological system. An important application of biomaterials is scaffolds for tissue engineering. One of the most common scaffolds is collagen-based scaffolds.

Collagen is the major structural component in mammalian tissues and mediates numerous biological processes. Its unique hierarchical self-assembling nature, together with high abundance in vivo, has made it a fascinating scaffold in the field of biomimetics. In addition to collagen, glycosaminoglycans (GAGs) also serve as an essential element in tissues. These highly negatively charged macromolecules regulate many important physiological processes and the mechanical characteristics of collagen-based scaffolds. However, collagen-GAG composites derived from natural materials have drawbacks. For example, the immunogenicity, lot-to-lot variability, and complex purification associated with natural materials. These drawbacks have hindered efforts to generate defined molecular tools for engineering extracellular environments.

This invention therefore seeks to provide improved collagen-scaffolds.

### SUMMARY OF THE INVENTION

In a first aspect, there is provided a collagen molecule comprising three chains wherein at least one chain comprises at least one repeating unit of the general formula:

(X1-X2-G)n;

wherein X1 or X2 can be any amino acid or amino acid derivative provided that at least one of X1 or X2 is a glycosaminoglycan moiety;
G is glycine; and
n is a positive integer and is at least 1.

In a second aspect, there is provided a collagen molecule as defined herein for use in therapy.

In a third aspect, there is provided a method of treating a patient in need of therapy, comprising administering a collagen molecule as defined herein.

In a fourth aspect, there is provided a collagen molecule as defined herein for use in a cosmetic product.

In a fifth aspect, there is provided a collagen molecule as defined herein for use in tissue engineering.

In a sixth aspect, there is provide a collagen molecule as defined herein for use in the construction of a scaffold.

In a seventh aspect, there is provided a composition comprising a collagen molecule as defined herein and one or more excipients.

In an eighth aspect, there is provided a collagen molecule as defined herein for use in glycosaminoglycans-based pharmaceutics.

In a ninth aspect, there is provided a collagen molecule as defined herein for use as biomaterials for tissue engineering.

In a tenth aspect, there is provided a collagen molecule as defined herein for use in drug delivery or wound healing.

### BRIEF DESCRIPTION OF THE DRAWINGS

The invention will be better understood with reference to the detailed description when considered in conjunction with the non-limiting examples and the accompanying drawings, in which:

FIG. 1 is comprised of FIGS. 1A and 1B and shows the schematic examples of the collagen molecules disclosed herein. FIG. 1A shows the schematic illustration of a collagen molecule with three chains, wherein one chain comprises of a repeating unit. FIG. 1B shows the schematic illustration of a collagen molecule with three chains, wherein two or three chains comprises of a repeating unit. FIG. 1 illustrates that the repeating unit may be the same or different, within a chain or between chains. FIG. 1 also illustrates that the repeating unit may be repeated the same or different number of times within a given chain or between chains.

FIG. 2 is comprised of FIGS. 2A and 2B. FIG. 2A shows the schematic representation of collagen-GAG peptides known in the art. FIG. 2B shows the schematic representation of collagen-GAG peptides of the present invention. The GAG moiety as shown in FIG. 2 may be any GAG moiety. The amino acid or amino acid derivative shown in FIG. 2 may be any amino acid or any amino acid derivative.

FIG. 3 shows the difference between a heterogeneous chain of GAG moieties and a homogenous collagen chain as disclosed herein comprising homogenous GAG moieties.

FIG. 4 shows examples of various GAG moieties that can be used in the present invention.

FIG. 5 is comprised of FIGS. 5A and 5B. FIG. 5A shows the schematic illustration of electrostatic interactions between CS-A disaccharide and positively charged amino acids in collagen mimetic peptides. FIG. 5B shows the structures of peptides used.

FIG. 6 is comprised of FIGS. 6A to 6F. FIG. 6A shows the CD spectra of peptide 1. FIG. 6B shows the CD spectra of peptide 2 and 3. FIGS. 6C and 6D show the thermal denaturation curves and first derivative plots versus temperature for 1:2 mixture of peptide 1•2. FIGS. 6E and 6F shows the thermal denaturation curves and first derivative plots versus temperature for 1:2 mixture of peptide 1•3.

FIG. 7 is comprised of FIGS. 7A and 7B and shows the CD spectra for 1:2 mixture of peptide 1•2 (FIG. 7A) and peptide 1•3 (FIG. 7B).

FIG. 8 is comprised of FIGS. 8A to 8D and shows the ¹H, ¹⁵N-HSQC spectra of the peptide 4 (FIG. 8A), 1:2 mixture of peptide 1•4 (FIG. 8B), 4:1 mixture of CS-A disaccharide and peptide 4 (FIG. 8C), DSC melting profile for the 1:2 annealed mixture of peptide 1•3 (FIG. 8D).

FIG. 9 is comprised of FIGS. 9A to 9B and shows the ¹H, ¹⁵N-HSQC spectra of the 4:1 (FIG. 9A), 10:1 mixture of CS-A disaccharide and peptide 4 (FIG. 9B).

FIG. 10 shows the analytical HPLC traces (top) and ESI mass data (bottom) of the peptide 1.

FIG. 11 shows the analytical HPLC traces (top) and ESI mass data (bottom) of the peptide 2.

FIG. 12 shows the analytical HPLC traces (top) and ESI mass data (bottom) of the peptide 3.

FIG. 13 shows the analytical HPLC traces (top) and ESI mass data (bottom) of the peptide 4.

FIG. 14 shows the analytical HPLC traces (top) and ESI mass data (bottom) of the peptide 5.

FIG. 15 shows the ¹H NMR spectra of the fully protected disaccharide 5, after conversion from trichoroacetimidate 4 in one embodiment of the method of synthesizing the GAG-containing collagen peptide 1 as defined above as depicted in the scheme 1.

FIG. 16 shows the ¹³C NMR spectra of the fully protected disaccharide 5, after conversion from trichoroacetimidate 4 in one embodiment of the method of synthesizing the GAG-containing collagen peptide 1 as defined above as depicted in the scheme 1.

FIG. 17 shows the ¹H NMR spectra of the acetamide 6 after radical-mediated reduction of N-trichloroacetyl group to N-acetyl congener with n-tributylstannane and AIBN in one embodiment of the method of synthesizing the GAG-containing collagen peptide 1 as defined above as depicted in the scheme 1.

FIG. 18 shows the ¹³C NMR spectra of the acetamide 6 after radical-mediated reduction of N-trichloroacetyl group to N-acetyl congener with n-tributylstannane and AIBN in one embodiment of the method of synthesizing the GAG-containing collagen peptide 1 as defined above as depicted in the scheme 1.

FIG. 19 shows the ¹H NMR spectra of the diol 7 after hydrolysis of the benzylidene acetal of 6 followed by removal of the TMS group in one embodiment of the method of synthesizing the GAG-containing collagen peptide 1 as defined above as depicted in the scheme 1.

FIG. 20 shows the ¹³C NMR spectra of the diol 7 after hydrolysis of the benzylidene acetal of 6 followed by removal of the TMS group in one embodiment of the method of synthesizing the GAG-containing collagen peptide 1 as defined above as depicted in the scheme 1.

FIG. 21 shows the ¹H NMR spectra of disaccharide 8 after the selective benzoylation of C6 hydroxyl group of diol 7 in one embodiment of the method of synthesizing the GAG-containing collagen peptide 1 as defined above as depicted in the scheme 1.

FIG. 22 shows the ¹³C NMR spectra of disaccharide 8 after the selective benzoylation of C6 hydroxyl group of diol 7 in one embodiment of the method of synthesizing the GAG-containing collagen peptide 1 as defined above as depicted in the scheme 1.

FIG. 23 shows the ¹H NMR spectra of the sulfated disaccharide motif after treatment of 8 with SO₃.trimethylamine complex in one embodiment of the method of synthesizing the GAG-containing collagen, peptide 1 as defined above as depicted in the scheme 1.

FIG. 24 shows the ¹³C NMR spectra of the sulfated disaccharide motif after treatment of 8 with SO₃.trimethylamine complex in one embodiment of the method of synthesizing the GAG-containing collagen peptide 1 as defined above as depicted in the scheme 1.

FIG. 25 shows the ¹H NMR spectra of the desired CS-A disaccharide 9 after sequential treat of LiOOH and NaOH in one embodiment of the method of synthesizing the GAG-containing collagen peptide 1 as defined above as depicted in the scheme 1.

FIG. 26 shows the ¹³C NMR spectra of the desired CS-A disaccharide 9 after sequential treat of LiOOH and NaOH in one embodiment of the method of synthesizing the GAG-containing collagen peptide 1 as defined above as depicted in the scheme 1.

### DETAILED DESCRIPTION OF THE PRESENT INVENTION

Collagen is the major structural component in mammalian tissues that mediates numerous biological processes. Currently, 28 different types of collagen are known, examples of these include but are not limited to collagen type I, collagen type II, collagen type III, collagen type IV and collagen type V. Of these, collagen type I is the most abundant in the body and is found in the skin. Collagen type I is also the most commonly used collagen in tissue engineering. Naturally occurring collagen has a triple helical structure made up of three protein strands or chains. It will be understood to one of skill in the art that different collagen types are made up of different types of protein strands. For example, collagen type I, IV and VIII are comprised of two α1 chains and one α2 chain, collagen type II, II VII and X are comprised of three α1 chains and collagen type VI is comprised of one α1, one α2 and one α3 chain. Modification to collagen molecules as disclosed herein can be done to one or more strands of the collagen molecule. In one example, one α1 chain of type VI collagen is modified. In another example, two α1 chains of type X collagen are modified.

It will also be understood to one of skill in the art that one distinctive feature of collagen is the presence of a repeating unit of amino acids in each chain or strand of the collagen molecule. This repeating amino acid unit is Gly-Pro-X or Gly-X-Hyp, where X is any amino acid and Hyp is hydroxyproline, a derivative of proline.

Collagen molecules may be modified or synthesized to impart desired characteristics such as stability, overall charge on one strand of a collagen molecule and interaction with other molecules.

Therefore, in a first aspect, the present invention provides a collagen molecule comprising three chains wherein at least one chain comprises at least one repeating unit of the general formula:

(X1-X2-G)n;

wherein X1 or X2 can be any amino acid or amino acid derivative provided that at least one of X1 or X2 is a glycosaminoglycan moiety;
G is glycine; and
n is a positive integer and is at least 1.

For example, n may be at least 2, at least 5, at least 10, at least 20, at least 50, at least 100, at least 200, at least 500 or at least 1000. In one example, n is between 1 and 10, between 2 and 5, between 2 and 4 or between 10 and 50. In one example, n is 4. Any range comprising any of the aforementioned values is also possible. Accordingly, it will be generally understood that there is no upper limit for n.

As used herein, the term “amino acid” refers to charged, uncharged, polar, non-polar, aromatic, non-aromatic and may include naturally occurring amino acids as well as non-naturally occurring amino acids. Naturally occurring amino acids, standard or canonical amino acids refer to the proteinogenic amino acids. Examples of these include but are not limited to the 20 essential amino acids such as, proline, valine, phenylalanine, glutamine, glutamic acid, aspartic acid, lysine, tyrosine and asparagine. Examples of non-naturally occurring amino acids, non-canonical amino acids or unnatural amino acids refer to amino acids other than the 20 essential amino acids, include but are not limited to non-proteinogenic amino acids, N-methyl amino acids, D-amino acids, diamino acids, proline and pyruvic acid derivatives, glycine derivatives and amino acids that have been modified.

As used herein, the term “amino acid derivative” refers to an amino acid that has been modified by the addition or removal of one or more functional groups or an amino acid, whose chirality has been modified. Examples of amino acid derivatives include but are not limited to hydroxyproline and protected amino acids such as Fmoc and Boc protected amino acids.

In one example of a collagen molecule disclosed herein, the collagen molecule has three chains wherein the at least one repeating unit is in one chain. It will be understood that the remainder of the chain comprising the repeating unit is comprised of amino acids or amino acid derivatives. It will also be understood that the remaining chains are comprised of amino acids, amino acid derivatives or mixtures thereof only. In another example of the collagen molecule disclosed herein, the collagen molecule has three chains wherein the at least one repeating unit is in two chains. It will be understood that the remainder of the chains comprising the repeating unit is comprised of amino acids or amino acid derivatives. It will also be understood that the remaining chain is comprised of amino acids, amino acid derivatives or mixtures thereof only. In yet another example of a collagen molecule disclosed herein, the collagen molecule has three chains where the at least one repeating unit is in all three chains. It will be understood that the remainder of the chains are comprised of amino acids or amino acid derivatives. Furthermore, it will be readily understood to one of skill in the art that the repeating unit within a chain may be different or that n may be different. It will also be readily understood to one of skill in the art that the repeating unit may be different between chains or that n may be different between chains. For example, in any given chain of one collagen molecule, a repeating unit may be repeated 5 times while a different repeating unit may be repeated 10 times while the first repeating unit or yet another repeating unit may be repeated 20 times in a second chain of the same molecule, and the third chain is comprised only of amino acids or amino acid derivatives.

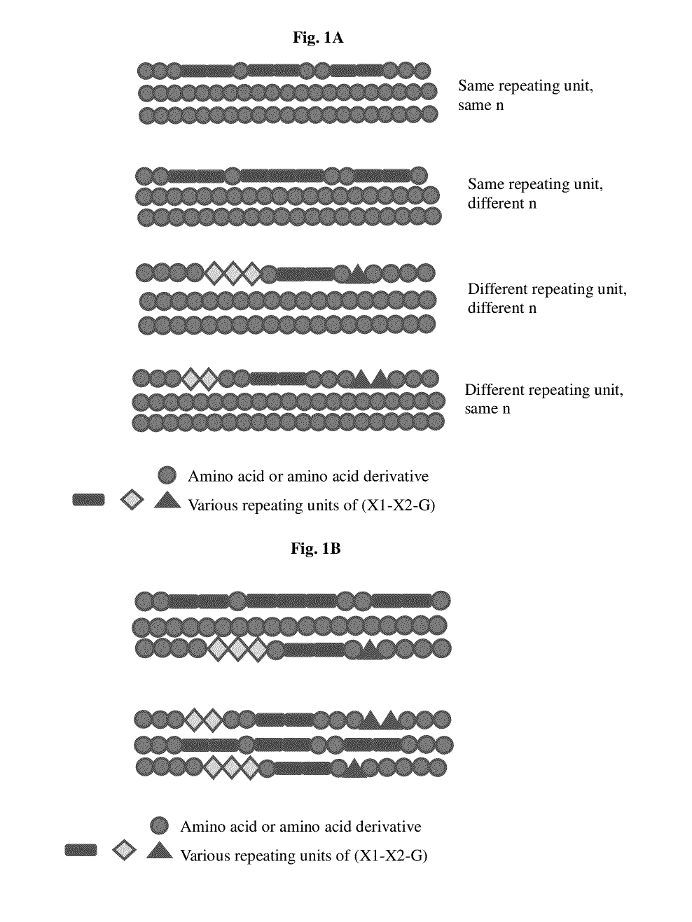

For the sake of clarity, examples of collagen molecules disclosed herein are schematically presented in FIG. 1. FIG. 1A shows examples of collagen molecules disclosed herein wherein the at least one repeating unit is on one chain. It will be understood from FIG. 1A that the repeating unit may be the same or different and that n may be the same or different. FIG. 1B shows examples of collagen molecules disclosed herein wherein the at least one repeating unit is on two or three chains. It will be understood that the repeating unit may be the same or different between chains. It will also be understood that n may be the same or different between chains. It will be understood that FIG. 1 represents examples of the collagen molecules disclosed herein and in no way limits the scope of the molecules claimed. As used herein, collagen may be native collagen or gelatinized collagen. Gelatinized collagen refers collagen that has been denatured, for example, by thermal or chemical means. Native collagen refers to collagen that has not been denatured. It will be generally understood that denaturation refers to the loss of the quaternary structure of a protein, such as collagen and may be reversible or irreversible.

As used herein, collagen may be obtained from natural sources or may be synthetic. Natural sources of collagen include but are not limited to animal tissue, for example, mammalian tissue and avian tissue.

Collagen derived from natural sources has a number of disadvantages, such as immunogenicity, lot-to-lot batch variation and requires complex purification which are not present in synthetic collagen. Accordingly, the present invention involves the use of synthetic collagen. Synthetic collagen includes but is not limited to collagen mimetic peptides and synthetic self-assembling collagen. It will be generally understood that synthetic self-assembling collagen refers to synthetic peptide chains that self-assemble in a way that mimics the self-assembly of natural collagen. For example, assembly of peptides into triple helices, assembly of triple helices into a fiber, and a hydrogel. An example of synthetic collagen is collagen mimetic peptides (CMP).

As used herein, the term “mimetic peptide” refers to synthetic peptides that have similar characteristics to a native peptide. Synthetic peptides will be generally understood to mean peptides which are man-made rather than isolated from animal sources. Characteristics that synthetic peptides have that are similar to native peptides include binding affinity to target proteins and mechanical properties. Synthetic peptides have a number of advantages, for example, synthetic peptides allow an amino acid sequence to be customized: Synthetic peptides are also distinguishable from native peptides based on the method of synthesis. For example, synthetic peptides may be synthesized by solid-phase peptide synthesis or solution-phase peptide synthesis.

Examples of mimetic peptides include but are not limited to collagen mimetic peptide and glycosaminoglycan (GAG)-mimetic peptide. Another example of a mimetic peptide is a collagen-GAG mimetic peptide.

It will be understood to one of skill in the art that mimetic peptides vary in length and that the length of mimetic peptides depend on the biological systems targeted. For example, mimetic peptides may range in length from at least 5, at least 10, at least 15, at least 20, at least 25, at least 30, at least 40, at least 50, at least 100, at least 500 and at least 1000 amino acids. For example, a mimetic peptide may be 24 amino acids long.

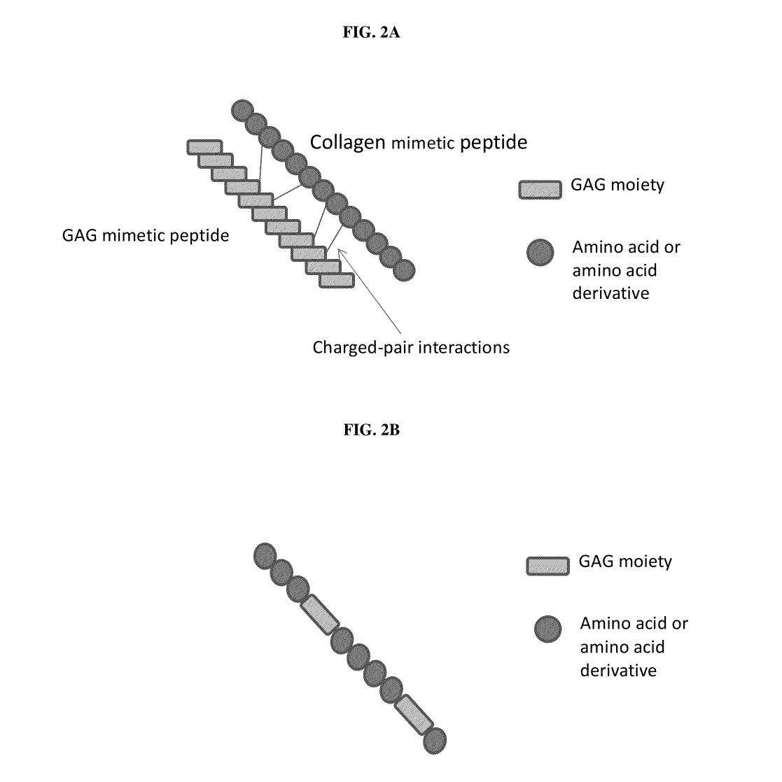

The present invention discloses a collagen-GAG mimetic peptide. The terms “collagen-GAG mimetic peptide” or “collagen-GAG composite” as is presently known in the art refers to a collagen-GAG mimetic peptide comprising a collagen mimetic peptide and a GAG mimetic peptide that interact with each other through charge-pair interactions (FIG. 2A). Collagen-GAG mimetic peptides or collagen-GAG composites as is known in the art are therefore comprised of one or more strands of a collagen peptide and one or more strands of a GAG peptide that are separate and distinct from each other.

However, the terms “collagen-GAG mimetic peptide” or “collagen-GAG composite” as used herein in reference to the invention refers to a collagen peptide comprising a GAG moiety within the collagen peptide (FIG. 2B). For example, a collagen-GAG mimetic peptide as disclosed herein has the general formula (X1-X2-G)n; wherein X1 or X2 can be any amino acid or amino acid derivative provided that at least one of X1 or X2 is a glycosaminoglycan moiety; G is glycine; and n is a positive integer and is at least 1. Therefore, the collagen-GAG mimetic peptides or collagen-GAG composites of the invention comprises a collagen peptide with GAG moieties incorporated into the same strand of the collagen peptide.

It will be readily understood by one of skill in the art that peptides derived from natural sources are heterogeneous. Accordingly, one advantage of the peptides provided herein is the ability to customize these such that they are homogeneous.

As used herein, the term “heterogeneous” refers to a peptide or peptide chain that has units that differ randomly. For example, a naturally-derived GAG chain will have randomly different disaccharide units. These disaccharide units may differ in terms of the presence or absence of one or more sulfate or carboxylate groups. The heterogeneity of peptides results in variability in the characteristics of the peptides. A disadvantage of this is the lot-to-lot and batch variability between the peptides. Accordingly, an advantage of the “collagen-GAG mimetic peptide” or “collagen-GAG composite” of the present invention is that these are homogeneous. As used herein, the term “homogeneous” means that the units of any given peptide chain are non-random. For example, a homogeneous collagen-GAG mimetic peptide or a homogenous collagen chain or strand as disclosed herein will have the same repeating unit that is repeated in a non-random pattern. For example, a homogeneous collagen-GAG mimetic peptide disclosed herein can also have different repeating units that repeats in a non-random pattern. The number of times a repeating unit is repeated, n, is also non-random.

As used herein, homogeneous may also be used to refer to a collagen molecule wherein at least one chain is homogenous. For example, a homogeneous collagen molecule with three chains may have one homogenous chain and two heterogeneous chains. For example, a homogenous collagen molecule may have two homogenous chains and one heterogeneous chain. In yet another example, a homogenous collagen molecule with three chains may have all three chains that are homogenous. It will be readily understood that the homogeneous chains in a homogeneous collagen molecule with two homogenous chains may be the same or different. It will also be readily understood that in a homogeneous collagen molecule with three homogenous chains, each of these chains may be different from each other or identical to each other. For example, a collagen molecule disclosed herein may be comprised of three chains, wherein at least one of the chains is an amino acid-GAG chain. This means that at least one chain is an amino acid-GAG chain and the other chain or chains are pure amino acid chains. Another example of a collagen molecule disclosed herein may be a collagen molecule comprised of three chains wherein each of the three chains is an amino acid-GAG chain. It will be clear to one of skill in the art that the repeating units in the amino acid-GAG chains may be the same or different.

The term “homogenous” as used herein may also refer to the homogeneity of the GAG moieties within a collagen-GAG peptide chain or within a repeating unit. For example, in the repeating unit (X1-X2-G)n, X1 is comprised of the same GAG moiety or X1 is one GAG moiety and X2 is a different GAG moiety. In yet another example; all the GAG moieties within a collagen-GAG chain are the same. It will be understood that the GAG moieties are non-random.

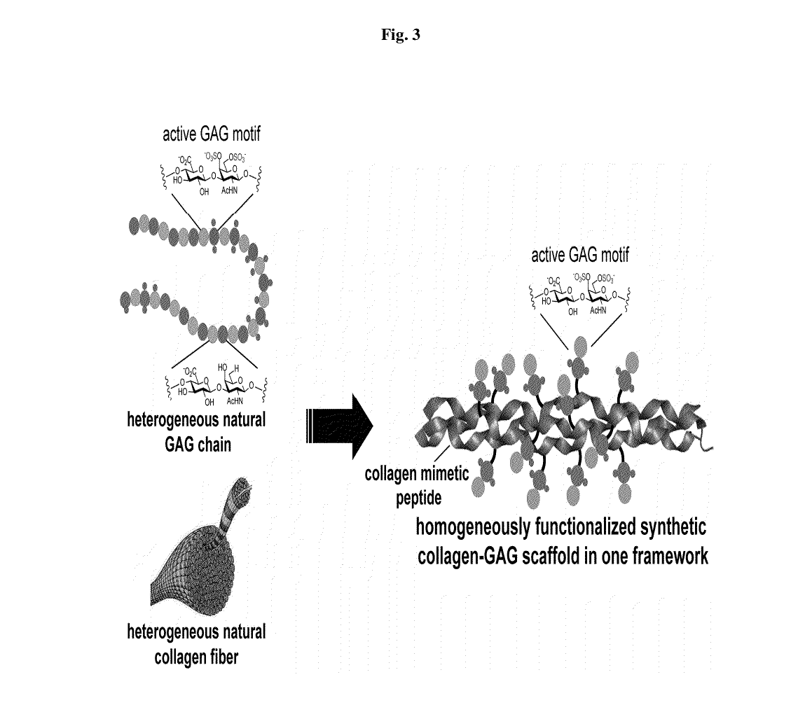

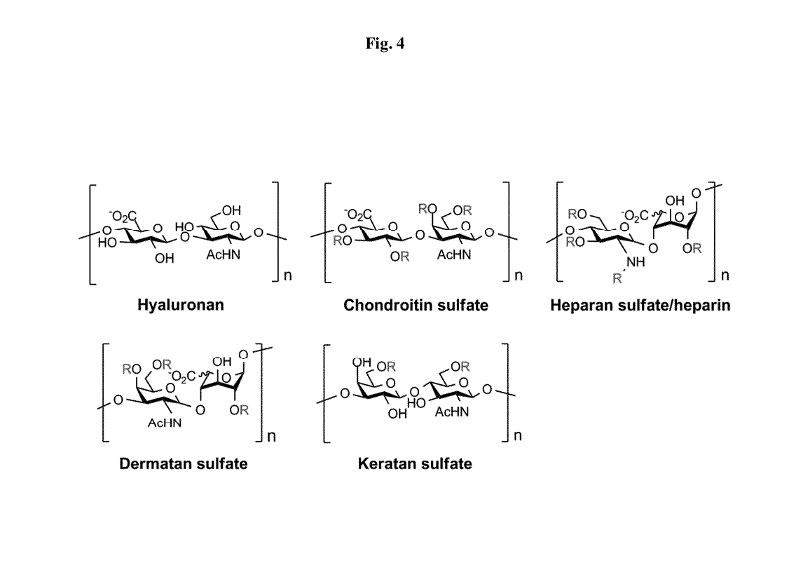

Accordingly, a homogeneous synthetic peptide chain may be represented as follows: A-B-B-B-A-B-B-B-A-B-B-B or B-A-B-A-B-A-B-A-B-A or A-A-A-B-A-A-A-B-A-A-A-B, where A may represent a repeating unit within any given peptide chain and B may represent any amino acid or amino acid derivative within the peptide chain. The representation shown above can also be applied to the GAG moieties within a repeating unit or within a collagen-GAG peptide chain, wherein A represents a GAG moiety and B represents a different GAG moiety. It will be clear to one of skill in the art that the above representations are merely schematic examples of a homogenous peptide chain. It will also be understood to one of skill in the art from this schematic representation that the units are not random. FIG. 3 illustrates one example of a homogenous collagen molecule as disclosed herein comprising homogenous, non-random GAG moieties. FIG. 3 provides one example of a homogenous collagen molecule wherein the GAG moieties of the collagen molecule are the same. FIG. 3 also illustrates a heterogeneous chain of GAG moieties. It is readily understood from FIG. 3 that the heterogeneous GAG moieties are random.

As already stated, the present invention provides a collagen molecule comprising three chains wherein at least one chain comprises at least one repeating unit of the general formula:

(X1-X2-G)n;

wherein X1 or X2 can be any amino acid or amino acid derivative provided that at least one of X1 or X2 is a glycosaminoglycan moiety;
G is glycine; and
n is a positive integer and is at least 1.

Accordingly, examples of a collagen-GAG mimetic peptide as disclosed herein include but are not limited to Ac-(PXG)₃-(CS-U-XG)₂-(PXG)₃-C(O)NH₂, Ac-(PXG)₃-(CS-A-XG)₂-(PXG)₃-C(O)NH₂ and Ac-(PXG)₂-(CS-A-XG)₄-(POG)₂-C(O)NH₂, wherein Ac is acetyl; P is proline; G is glycine; CS-U is unsulfated chondroitin sulfate; CS-A is chondroitin sulfate A; K is lysine; R is arginine; and X is hydroxyproline.

The modification or synthesis of a collagen molecule may be may be undertaken to impart desired characteristics that are not present in the unmodified molecule or that are not present in the natural molecule. For example, a collagen molecule may be modified to improve the stability between the strands of the collagen triple helix. As used herein, the term “modified”, “modification”, “modifying” and other grammatical variants thereof refer to the alteration of collagen. An example of modification of a collagen molecule is the addition of compounds to one or more strands of a collagen molecule or to the overall collagen molecule. For example, the addition of a carbohydrate to one strand of a collagen molecule. The addition of carbohydrates to one or more strands of a collagen molecule may be added to modify the charge of one or more strands of a collagen molecule. In particular, the addition of carbohydrate molecules on to one or more strands of a collagen molecule imparts a negative charge onto a collagen molecule.

Other examples of modification include but are not limited to the alteration of the charge on one or more strands of a collagen molecule, alteration of the overall charge of a collagen molecule, alteration of the physical and chemical characteristics of a collagen molecule, for example, melting temperature, thermal stability, protein conformation and light absorption. Examples of modification also include substitution of one or more strands of a collagen triple helix, modification of one or more amino acids in one or more strands of a collagen triple helix molecule, modification of the interaction between one or more strands of a collagen molecule or between collagen molecules, acetylation of one or more strands of a collagen molecule and amidation of one or more strands of a collagen molecule. Modification of one or more strands of a collagen triple helix molecule may result in a collagen heterotrimer or a collagen homotrimer.

As already mentioned, collagen has a triple helical structure. As used herein, the term “heterotrimer” in reference to a collagen molecule means that the three strands are not identical. For example, in type I collagen which is comprise of one α2 chain and two al chains, the α2 chain may be different to the two α1 chains, the α2 chain and one of the al chain are different to the third α1 chain or each of the chains are different to the other. Other examples of collagen heterotrimers include but are not limited to collagen type IV and VIII which comprise of one α2 chain and two α1 chains. These types of collagen are also known as heterotrimeric AAB collagen. Yet other examples of heterotrimeric collagen include but are not limited to collagen type VI which comprises of one α1, one α2 and one α3 chains. This type of collagen molecule is also known as heterotrimeric ABC collagen.

As used herein, the term “homotrimer” in reference to a collagen molecule means that each of the a-chains of the collagen molecule is identical to each other. For example, collagen type II, III, VII and X comprise three α1 chains and are homotrimers. An example of a modified collagen molecule is a collagen molecule wherein one chain is positively charged and the other two chains are negative charged. Another example of a modified collagen molecule is a collagen molecule wherein one chain is negatively charged and the other two chains are positively charged. Yet another example of a modified collagen molecule is a collagen molecule has a net-neutral charge.

As used herein, the term “net-neutral” means that the overall charge of a collagen molecule is neutral.

Charge neutrality may be achieved for example, by a first α2 collagen chain that is positively charged, and a second and third α1 collagen chains that are both negatively charged, by a first α2 collagen chain is negatively charged, and a second and third al collagen chains that are both positively charged, by a first α2 collagen chain, a second al collagen chain, and/or a third α1 collagen chain comprising one or more carbohydrate molecules or by a first α2 collagen chain, a second α1 collagen chain and/or a third al collagen chain that comprises one or more positively or negatively charged amino acid residues. An example of a modified collagen molecule is a collagen molecule wherein at least one of the amino acids within one or more strands has been substituted with a positively charged amino acid. Examples of positively charged amino acids include but are not limited to aspartic acid, glutamic acid, lysine, arginine, and histidine. An example of a modified collagen molecule is a collagen molecule wherein one or more amino acids on one or more strands of the collagen molecule is substituted with lysine or arginine.

An example of a modified collagen molecule is a collagen molecule wherein an amino acid on one strand of the triple helix has been substituted with lysine, wherein an amino acid on one strand of the triple helix has been substituted with arginine and wherein a carbohydrate has been added onto the third strand of the triple helix. Another example of a modified collagen molecule is a collagen molecule wherein an amino acid has been substituted with lysine on two strands of the triple helix and wherein a carbohydrate has been added onto the third strand of the triple helix. Another example of a modified collagen molecule is a collagen molecule wherein an amino acid has been substituted with arginine on two strands of the triple helix and wherein a carbohydrate has been added onto the third strand of the triple helix. Another example of a modified collagen molecule is a glycosaminoglycan-containing collagen mimetic peptide.

Yet another example of a modified collagen molecule is collagen molecule wherein an amino acid within one or more strands of a collagen molecule is modified by radioactive labeling. An example of such a modified amino acid is ¹⁵N-enriched glycine. A radioactively labeled amino acid on a collagen molecule may be used to determine the physical characteristics of a collagen molecule, for example, the heterotrimeric formation of a collagen molecule. For example, nuclear magnetic resonance (NMR) may be used to determine the heterotrimeric formation of a ¹⁵N-enriched glycine labeled collagen molecule.

As stated herein, a collagen molecule may be modified by the incorporation of a GAG moiety within at least one chain of a collagen molecule. Addition of a GAG moiety within one or more strands of the collagen triple helix confers stability onto the molecule due to electrostatic charge pair interactions between the negatively charged GAG moiety with positively charged amino acids within the same strand of the collagen triple helix or between one or more strands of the collagen triple helix. The positively charged amino acids may be naturally occurring within a collagen strand or may be the result of substitution of one or more amino acids on one or more strands of a collagen molecule with a positively charged amino acid. As used herein, “glycosaminoglycan” (GAG) or “GAG moiety” refers to unbranched polysaccharides consisting of a repeating disaccharide unit. Glycosaminoglycans are generally understood to be one of the major structural components in tissues and are an essential element in tissues.

As used herein, “disaccharide” refers to a molecule that has two monosaccharide units. It will be understood to one of skill in the art that the minimum unit of a GAG is a disaccharide. It will also be understood to one of skill in the art that a GAG may be represented as follows: (disaccharide)n, wherein n can be any integer. It will also be clear to a skilled person that the repeating disaccharide unite in a GAG may be the same or may be different.

Examples of GAGs include but are not limited to heparan sulfate (heparin), chondroitin sulfate, keratan sulfate, dermatan sulfate and hyaluronic acid (hyaluronan). An example of a GAG is chondroitin sulfate. Examples of chondroitin sulfate include but are not limited to chondroitin sulfate A (CS-A), chondroitin sulfate C (CS-C), chondroitin sulphate D (CS-D), chondroitin sulphate E (CS-E), chondroitin sulfate U (CS-U), chondroitin sulfate K (CS-K), chondroitin sulfate L (CS-L) and unsulfated chondroitin. An example of chondroitin sulfate is chondroitin sulfate A or chondroitin sulfate U. Examples of various types of GAG are shown in FIG. 4.

As disclosed herein, the incorporation of a GAG moiety into at least one chain of a collagen molecule may be achieved by any coupling reaction. Coupling reactions will be understood by a person of skill in the art. For example, a GAG moiety may be incorporated into at least one chain of a collagen molecule by a click reaction, amidation or aldonation.

Glycosaminoglycans (GAG) may be “alkyne-functionalised” to allow covalent modification of the GAG. As used herein, the term “alkyne-functionalised” refers to the addition of alkyne functional groups onto a molecule. The alkyne functional group may be added to the terminal carbon or an internal carbon. An example of a class of molecules that may be alkyne-functionalised is carbohydrates, for example, chondroitin sulfate A. Alkyne-functionalised chondroitin sulfate A may be synthesised by:

converting tricholoroacetimidate 4

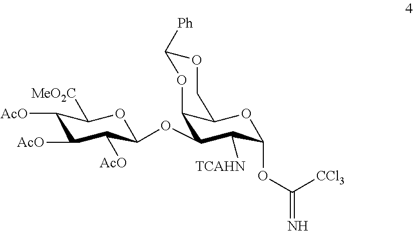

to the fully protected disaccharide 5

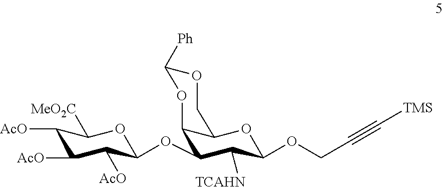

using trimethylsilyl triflate; reducing N-trichloroacetyl group to N-acetyl cogner with n-tributylstannane and AIBN to yield the acetamide 6

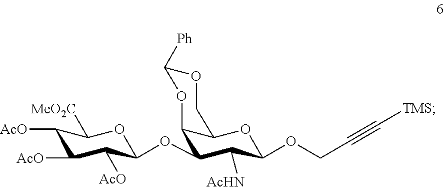

hydrolysing the benzylidene acetal followed by deprotecting the TMS group to produce the diol 7

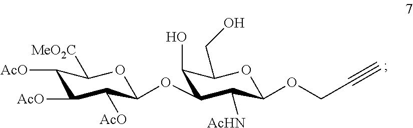

selectively benzoylating the C6 hydroxyl group using benzoyl cyanide to yield agent 8

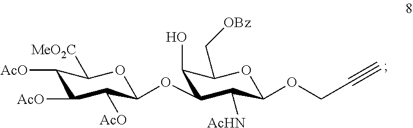

treating The agent 8 with SO₃.trimethylamine complex; and treating the resultant mixture with LiOOH and NaOH.

An alkyne functional group may be protected. As used herein, “protection”, “protecting”, “protective” or “protected” or grammatical variant thereof refers to the addition of a functional protecting group into a molecule. The addition of a protective group provides chemoselectivity in a subsequent chemical reaction or protects a molecule in a given reaction from specific reaction conditions. Chemoselectivity refers to the preferential reactivity of a functional group. An alkyne functional group may be deprotected. As used herein, “deprotection”, “deprotected” and grammatical variants thereof refer to the removal of a functional group in a molecule.

Alkyne-functionalised molecules are particularly useful in click reactions wherein the functionalised molecule can be “clicked” to another target molecule. For example, an alkyne functionalised carbohydrate such as chondroitin sulphate A (CS-A) may undergo a click reaction to be added to a collagen peptide or molecule.

As used herein, “click chemistry” or “click reaction” refers to the synthesis of compounds via heteroatom links (C-X-C) first described by K. B. Sharpless. It will be understood to one of skill in the art that reaction conditions will vary depending on the start and end products. For example, a click reaction may be carried out in a solvent at ambient temperature in the presence of copper iodide, a base and tris[(1-benzyl-1H-1,2,3-triazol-4-yl)methyl]amine (TBTA) under argon atmosphere. An example of a base is N,N-diisopropylethylamine (DIPEA). An example of a solvent is dimethyl sulfoxide (DMSO). An example of a click reaction is a reaction wherein one or more carbohydrate molecules is incorporated into a collagen molecule.

As used herein, the term “ambient temperature” refers to the temperature of an environment. It will be understood to one of skill in the art that ambient temperature refers to room temperature. It will also be understood to one of skill in the art that ambient temperature or room temperature includes a range of temperature, for examples, from about 5° C. to about 50° C. For example, from about 5° C. to about 45° C., from about 5° C. to about 40° C., from about 5° C. to about 30° C., from about 5° C. to about 29° C., from about 5° C. to about 28° C., from about 5° C. to about 27° C., from about 5° C. to about 26° C., from about 5° C. to about 25° C., from about 5° C. to about 24° C., from about 5° C. to about 23° C., from about 5° C. to about 22° C., from about 5° C. to about 21° C., from about 5° C. to about 20° C., from about 5° C. to about 19° C., from about 5° C. to about 18° C., from about 5° C. to about 17° C., from about 5° C. to about 16° C., from about 5° C. to about 15° C., from about 5° C. to about 14° C., from about 5° C. to about 13° C., from about 5° C. to about 12° C., from about 5° C. to about 11° C., from about 5° C. to about 10° C., from about 5° C. to about 9° C., from about 5° C. to about 8° C., from about 5° C. to about 7° C., from about 5° C. to about 6° C.

As stated earlier, examples of a collagen-GAG mimetic peptide as disclosed herein include but are not limited to Ac-(PXG)₃-(CS-U-XG)₂-(PXG)₃-C(O)NH₂, Ac-(PXG)₃-(CS-A-XG)₂-(PXG)₃-C(O)NH₂ and Ac-(PXG)₂-(CS-A-XG)₄-(POG)₂-C(O)NH₂, wherein Ac is acetyl; P is proline; G is glycine; CS-U is unsulfated chondroitin sulfate; CS-A is chondroitin sulfate A; K is lysine; R is arginine; and X is hydroxyproline.

Therefore, an example of a collagen molecule, is a collagen molecule wherein (CS-A-XG) has the following formula:

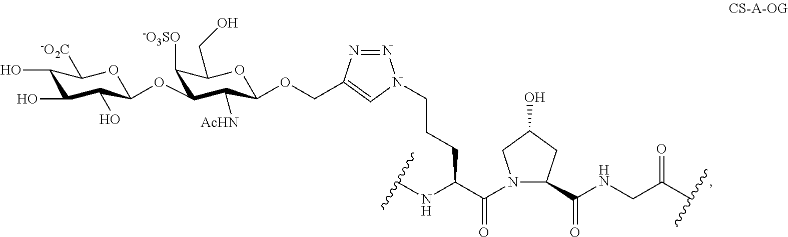

and wherein (PXG) has the following formula:

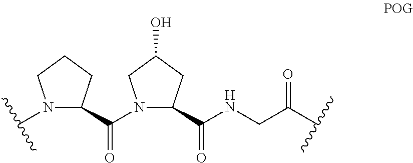 [SMILES: *C(=O)CNC(=O)[C@@H]1C[C@@H](O)CN1C(=O)[C@@H]1CCCN1*]

An example of a modified collagen molecule is a collagen molecule wherein the one collagen chain has the formula Ac-(PXG)₂-(CS-A-XG)₄-(POG)₂-C(O)NH₂, and the second and third collagen chains have the formula Ac-(PXG)₂-(PRG)₄-(PXG)₂-C(O)NH₂.

Another example of a modified collagen molecule is a collagen molecule wherein one collagen chain has the formula Ac-(PXG)₂-(CS-A-XG)₄-(POG)₂-C(O)NH₂, and the second and third collagen chains have the formula Ac-(PXG)₂-(PKG)₄-(PXG)₂-C(O)NH₂.

As disclosed herein, a collagen molecule may be modified to improve stability.

As used herein, the term “stability”, “stabilized”, “stabilization” or grammatical variants thereof in reference to a collagen molecule refers to the ease in which the strands of the collagen triple helix unfolds. Stability may be measured based on melting temperature (Tm) wherein the Tm refers to the temperature at which 50% of the strands of a collagen molecule dissociates. Accordingly, a higher Tm indicates a higher stability of the molecule. For example, a collagen molecule with a Tm of 42.3° C., 42.3° C. or 41.3° C. is more stable than a collagen molecule with a Tm of 39.4° C., which is in turn more stable than a collagen molecule with a Tm of 16.9° C. or 17.4° C. Stability may also be measured using circular dichroism (CD) or differential scanning calorimetry (DSC). Circular dichroism refers to the differential absorption or left and right circularly polarized light. CD spectroscopy performed at varying temperatures provide information about the helical profile of collagen peptide. For example, a collagen peptide that maintains its polyproline type II helical profile as determined by CD spectroscopy at 25° C. is more stable than a collagen peptide that displays random coil conformations at 25° C. DSC refers to the method of measuring stability of a protein in which the difference in the amount of heat required to increase the temperature of a sample and reference is measured. For example, a collagen peptide which requires a higher amount of heat to increase its temperature is more stable than one that requires less heat.

It will be generally understood that there is no absolute temperature or criteria to determine stability of a peptide. Rather, the temperature or criteria in which stability is determined will be understood to be based on the target biological system.

Stability of a collagen molecule may be conferred by complementary electrostatic interactions between the strands of a collagen molecule. Accordingly, a method of improving the stability of a collagen molecule is also provided, wherein the collagen molecule is stabilised by the charge-pair interactions between the one or more GAG moieties and the positively or negatively charged amino acid residues or charge-pair interactions between the one or more GAG moieties and the positively charged amino acid residues. As disclosed herein, modified collagen molecule may be derived from natural or synthetic sources. Accordingly, the method of improving the stability of a collagen molecule as provided by the invention further comprises preparing the peptide derivatives on solid support using an amide resin, applying Fmoc chemistry and cleaving the desired collagen molecules from the resin with simultaneous deprotection by treatment with a trifluoroacetic acid/water/triisopropylsilane mixture.

Peptides and their derivatives may be synthesised using Fmoc chemistry. As used herein, the term “Fmoc” refers to fluoroenylmethyloxycarbonyl chloride and “Fmoc chemistry” refers to solid phase peptide synthesis (SSPS) using Fmoc protecting groups to protect the a-amino groups. Repeated cycles of coupling-wash-deprotection-wash results in a growing peptide chain wherein the free N-terminal amine of a solid-phase attached peptide is coupled to a single N-protected amino acid unit which is then deprotected, revealing a new N-terminal amine to which a further amino acid may be attached. The growing peptide chain is immobilized on a solid support.

As already stated earlier, “protection” and “deprotection” refer to the addition or removal of functional groups. In reference to peptide synthesis, functional groups that may be used in protection and deprotection include but are not limited to acetyl groups, Fmoc groups, Boc groups, benzoyl, methyl ester groups, benzyl, carbamate, ketals and dithianines. As used herein, the term “solid support” refers to the structure on which peptides are immobilised to during solid phase peptide synthesis. A solid support provides an anchor for the growing peptide molecule. It will be understood in the art that a solid support may have different physical characteristics and selection of a solid support will be determined by the peptide to be synthesized and peptide synthesis reaction conditions. Examples of solid supports include but are not limited to polystyrene resin, polyacrylamide resin, polyethylene glycol resin, composites of the above, cellulose fibres, glass, gel-type polymers. An example of a solid support is amide resin.

The peptides synthesized herein undergo click reaction to incorporate the carbohydrate GAG moiety and the reaction is monitored by reverse phase HPLC. The reaction mixture is precipitated from Tetrahydrofuran/methanol, converted into their sodium salt form and the salt is purified by size-exclusion chromatography.

As used herein, the term “chromatography” refers to the technique of separating mixtures. It will be understood to one of skill in the art that chromatographic separation includes but is not limited to gas chromatography, liquid chromatography, affinity chromatography, ion exchange chromatography, size exclusion chromatography and reversed phase chromatography. An example of chromatography is size exclusion chromatography.

The collagen molecules disclosed herein may be used in therapy. Accordingly, the invention provides a method of treating a patient in need of therapy, comprising administering a collagen molecule as disclosed herein.

As used herein, the terms “therapy”, “treatment” or grammatical variants thereof refers to the alleviation of symptoms associated with a condition.

As used herein, the terms “administration” or “administer” refer to the delivery of a modified collagen molecule or a pharmaceutically acceptable salt thereof or of a pharmaceutical composition containing modified collagen molecule or a pharmaceutically acceptable salt thereof of this invention to an organism for the purpose of wound healing, drug delivery and therapy.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. Alternatively, one may administer the collagen molecule in a local rather than systemic manner, for example, via injection of the compound directly into a tissue.

The collagen molecules disclosed herein may also be used in a cosmetic product. As used herein, the term “cosmetic product” refers to a product that may be applied for altering the appearance of a subject without affecting the subject's body structure or function. It will be generally understood that a cosmetic product has no medical effect such as healing or treatment of a subject. For example, a cosmetic product may be used to reduce the appearance of a wrinkle, blemish or scar.

The collagen molecules disclosed herein may also be used in tissue engineering. As used herein, the term “tissue engineering” refers to the synthesis of tissues in vitro. Tissue engineering may be used to synthesise tissues for the replacement or repair of tissues. For example, tissue engineering may be used to synthesise collagen, bone, blood vessels, skin and cartilage. An important component of tissue engineering are scaffolds. Tissue engineering is most commonly used to synthesise skin. Accordingly, the collagen molecule disclosed herein may be used in skin tissue engineering.

Accordingly, the collagen molecules disclosed herein may also be used in the construction of a scaffold. As used herein, the term “scaffold” refers to a structure that provides support for cells attachment. A scaffold may also refer to a 3-dimensional structure that mimics tissue such as the extracellular matrix. Scaffolds may be derived from natural sources or may be synthetic and may be ceramics, synthetic polymers or natural polymers. Scaffolds may also be composites comprised of different types of biomaterials. Examples of scaffolds include but are not limited to polystyrene, poly-L-lactic acid, polyglycolic acid, collagen and collagen composites. An example of a collagen-composite scaffold is collagen-glycosaminolycan composite scaffold.

The collagen molecules disclosed herein may also be used as biomaterials for tissue engineering. As used herein, the term “biomaterials” refers to a substance, structure or surface that interacts in a biological system. Biomaterials may be obtained from natural sources or may be synthetic. Examples of biomaterials include but are not limited to hydrogels, polymers and ceramic. An example of a biomaterial is a collagen-based scaffold.

The present invention also provides a composition comprising a collagen molecule as disclosed herein and one or more excipients.

As used herein, “composition” or “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

Compositions of the present invention may be manufactured by processes well known in the art, e. g., by means of conventional mixing, dissolving, granulating, drageemaking, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols.

The collagen molecules disclosed herein may also be used in glycosaminoglycan-based pharmaceutics. As used herein, the term “glycosaminoglycan-based pharmaceutics” refers to the use of glycosaminoglycan-based compounds in the development of medicines, drugs and pharmaceutical compounds.

Accordingly, the present invention also discloses a collagen molecule as disclosed herein for use in drug delivery. As used herein, the term “drug delivery” refers to the administration of a composition, compound, pharmaceutical, drug or medicament via a suitable route as defined herein.

As collagen is the major protein in the fibrous tissue such as skin, the collagen molecules disclosed herein may also be used in wound healing.

As used herein, the term “wound healing” refers to the repair of trauma to the skin or connective tissue. A wound may be due to injury, for example, due to surgery, puncture, cut, burn or tear. Accordingly, the collagen molecules as disclosed herein may be used in skin replacement and skin repair. Skin replacement and skin repair may be cosmetic or may be therapeutic.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Non-limiting examples of the invention and comparative examples will be further described in greater detail by reference to specific Examples, which should not be construed as in any way limiting the scope of the invention.

### EXAMPLE 1

This example describes the design of glycosaminoglycan (GAG)—containing collagen mimetic peptides (CMP).

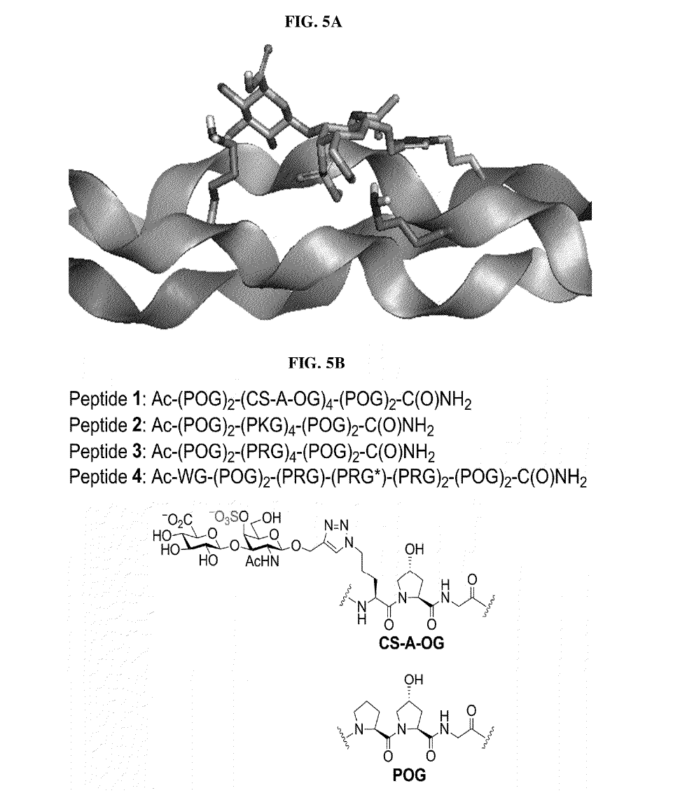

The GAG-containing CMP was designed to incorporate chondroitin sulfate (CS) A disaccharide motifs into a peptide chain. This CS-A motif provides unique biocharacteristics to cartilage tissues, including the mechanical properties of joints, the modulation of protein functions, and the proteolytic resistance of, collagen fibers. Sequence specific peptides consisting of two different Gly-Xaa-Yaa domains were hence designed. These peptides contained distinct repeating triplets with charged residues at the central core of peptide chains (FIG. 5B). CS-A disaccharide units were introduced at position Xaa (1) and either Lys or Arg at position Yaa (2 and 3) based on the positional preferences of charged residues in Gly-Xaa-Yaa sequence. The complementary electrostatic interactions between the negatively charged groups and basic residues present in two different chains would facilitate the formation of heterotrimeric helices over homotrimeric assembly (FIG. 5A). Peptide 4 containing ¹⁵N-enriched glycine was also designed for the compositional analysis of collagen triple helices (CTHs) using nuclear magnetic resonance (NMR) spectroscopy. In all cases, the C- and N-termini have been amidated or acetylated to avoid interactions with side chains (FIG. 5B).

### EXAMPLE 2

This example describes the synthesis of peptides.

An alkyne-functionalized CS-A disaccharide unit was first synthesized. Briefly, after converting 4 to a fully protected disaccharide 5 containing TMS protected alkyne, N-trichloroacetyl group was reduced to N-acetyl cogner with n-tributylstannane and -2,2′-Azobis(2-methylpropionitrile) (AIBN). Hydrolysis of the benzylidene acetal 6 followed by deprotecting TMS group afforded the diol 7. With the key intermediate in hand, the, synthesis was continued with the selective benzoylation of C6 hydroxyl group using benzoyl cyanide to yield agent 8. The desired CS-A disaccharide 9 was successfully elaborated by treating SO₃.trimethylamine complex followed by sequential treatment of LiOOH and NaOH (Scheme 1A).

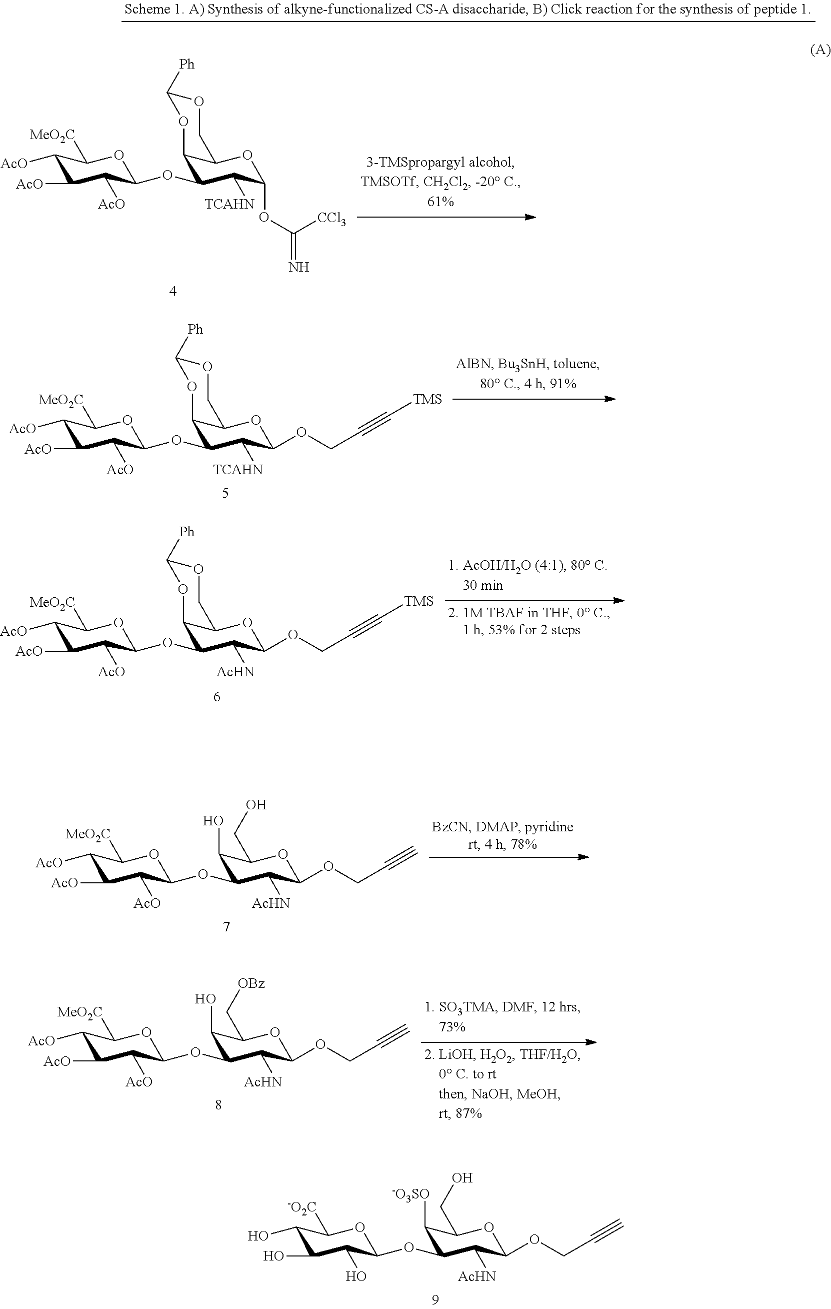

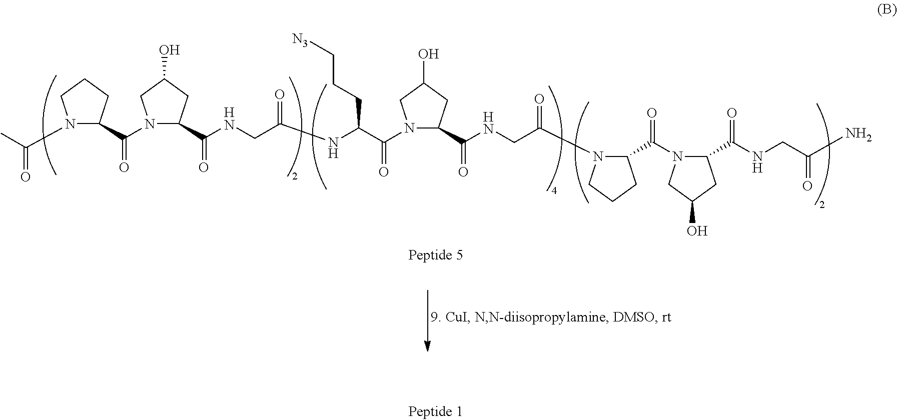

All peptide derivatives were prepared by standard Fmoc chemistry on solid support. The desired peptides were cleaved from the resin with a Trifluoroacetic acid/Water/Triisopropylsilane (TFA/H₂O/TIPS) cocktail [, purified to homogeneity by reverse phase HPLC, and characterized by mass spectroscopy. The click reaction was conducted in the presence of copper(I) diode and the consumption of azidopeptide was monitored by analytical reverse phase HPLC. The resulting reaction mixture was precipitated from Tetrahydrofuran/methanol (THF/methanol) and converted to its sodium salts. Purification by size-exclusion chromatography furnished the desired peptide 1 containing CS-A disaccharide units (Scheme 1B).

### EXAMPLE 3

This example describes the characterisation of the synthesised peptides.

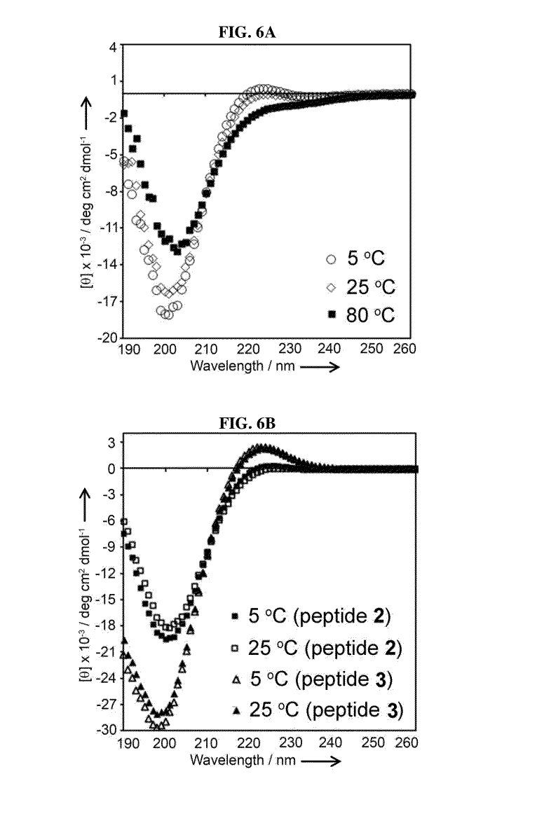

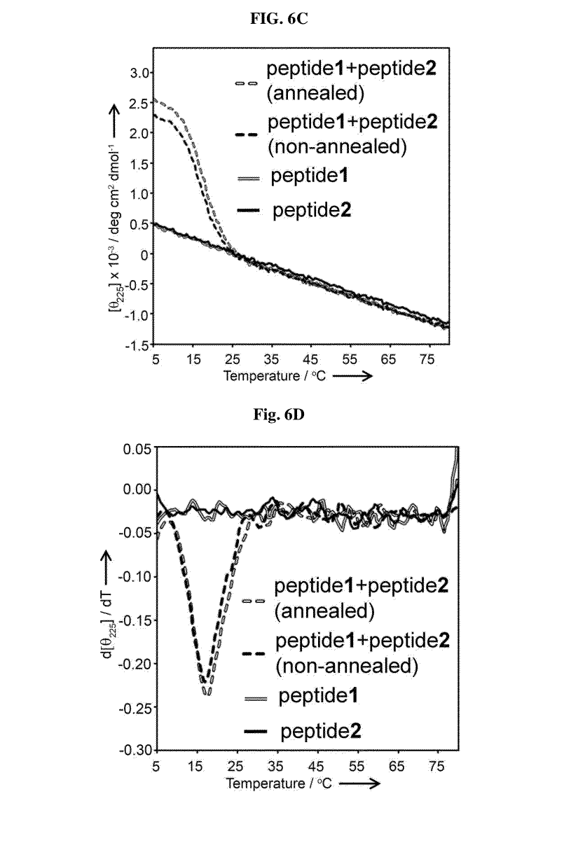

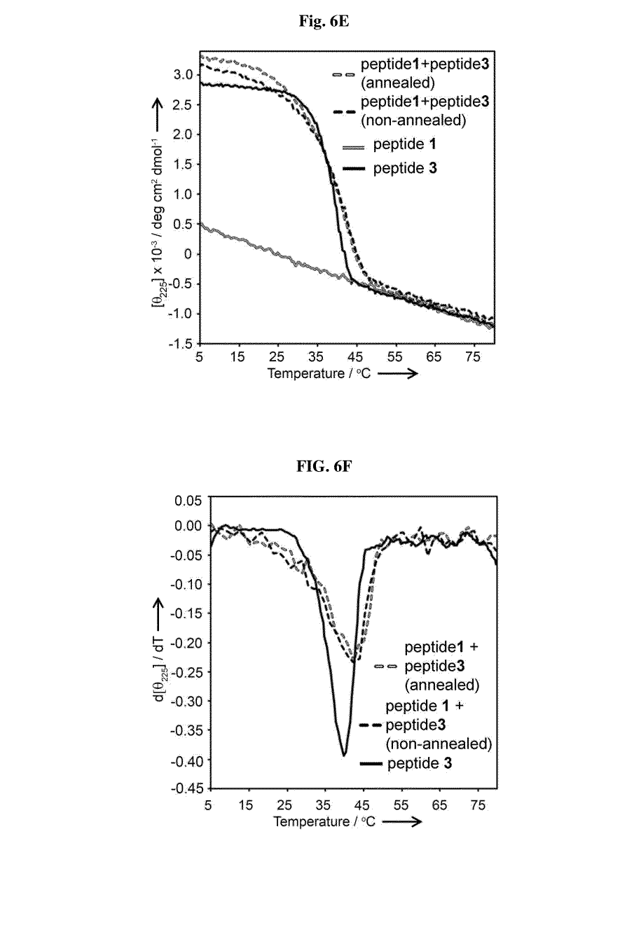

With the peptides successfully prepared, circular dichroism (CD) studies were carried out to characterize their homotrimeric properties. CD traces of all three peptides at 5° C. displayed polyproline type II (PPII) helical profiles with a maximum positive band at 224-226 nm and a minimum negative band at 198-201 nm. However, at 25° C., while peptide 3 retained the PPII helical structure, both 1 and 2 exhibited random coil conformations with only negative minimum peaks around 202 nm (FIGS. 6A and 6B). Thermal unfolding experiments were further performed in a range of 5° C. to 80° C. Both 1 and 2 turned out to be weak PPII helices or disordered structures with linear decreases in ellipticity, presumably due to the interhelical electrostatic repulsion between charged residues (FIGS. 6C and 6D). On the other hand, a cooperative triple helix unfolding for peptide 3 was observed with a Tₘ value of 39.4° C. (FIGS. 6C and 6D). This result is supported by the previous studies in which Arg side chains interact with backbone carbonyl groups in adjacent chains, thereby enhancing the triple helix stability.

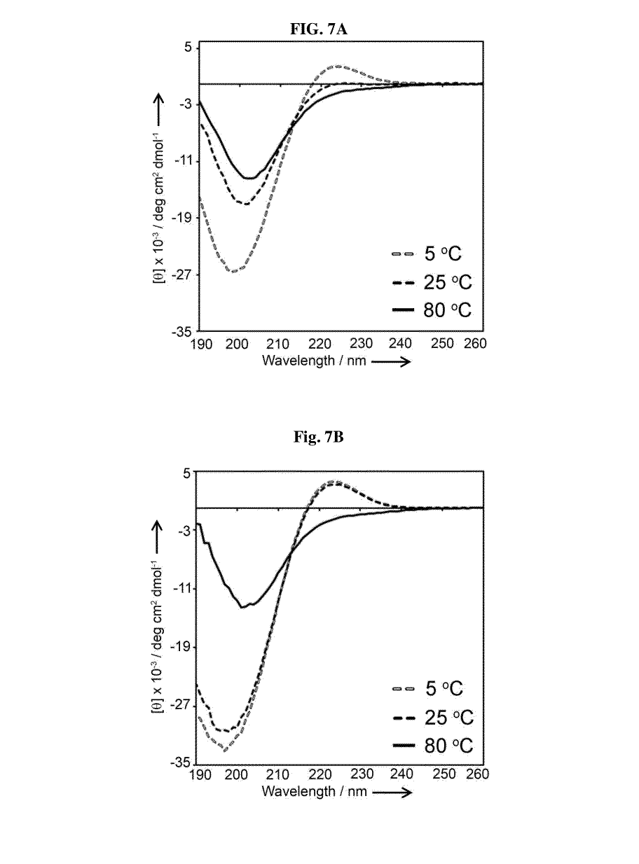

The heterotrimeric assembly of 1.2 with a molar ratio of 1:2 to make the overall charge neutral was next evaluated. A typical PPII helical conformation was identified at low temperature with a maximum positive at 224 nm and minimum negative band at 198 nm (FIG. 7). Thermal unfolding experiments indicated the existence of dominant heterotrimeric species with a Tₘ of 16.9° C. as the first derivative of unfolding curve displayed a single transition.

Annealing was also performed to drive the most thermodynamically favorable triple helix by dissociating any kinetically trapped species, and the identical unfolding profile was observed with the similar Tₘ of 17.4° C. to non-annealed trimer. These results demonstrated that negatively charged CS-A disaccharides were well accommodated in a CTH strand through the interactions with Lys residues in adjacent chains, while both individual peptide components did not form homotrimers. This result is significant considering the size of pendant sugars, as the replacement of even single proline residue with a bulky unit can cause the destabilization of CTH. However, despite demonstrating this heteromeric assembly, the low thermal stability would limit its practical applications.

Therefore investigated the combination of 1.3 containing Arg residues was investigated. A typical PPII helical conformation was identified for this mixture as expected from the result of Lys combination (FIG. 7B). Thermal unfolding experiments were further performed with/without preheating and the identical single cooperative transition curves were observed in both cases with Tₘ values of approximately 42° C. (FIGS. 6E and 6F), which are slightly higher than the Tₘ of homotrimer 3. In addition, the observed mean residual ellipticity (MRE) was nearly 40% higher than the weighted average of 1•3, suggesting the conversion of 1 from a disordered to an organized PPII helical conformation (FIG. 6E). Notably, Tₘ of 1.3 was markedly higher than that containing Lys residues by 24.9° C. of ΔTₘ. This result highlights the significance of sulfate-guanidinium interactions in stabilizing CTH, as Tₘ values of (EOG)₁₀.2(PRGPOG)₅ and (EOG)₁₀.2(PKGPOG)₅ mixtures are marginally different in neutral phosphate buffer by only 7.5° C. Nevertheless, the lack of compositional-information had limited ability to confirm the heterotrimeric assembly as CD studies provide only general idea for the folding and stability of assembled peptides. In particular, the melting curve of peptide 3 was quite similar to the mixture of 1.3, making it difficult to distinguish from one another.

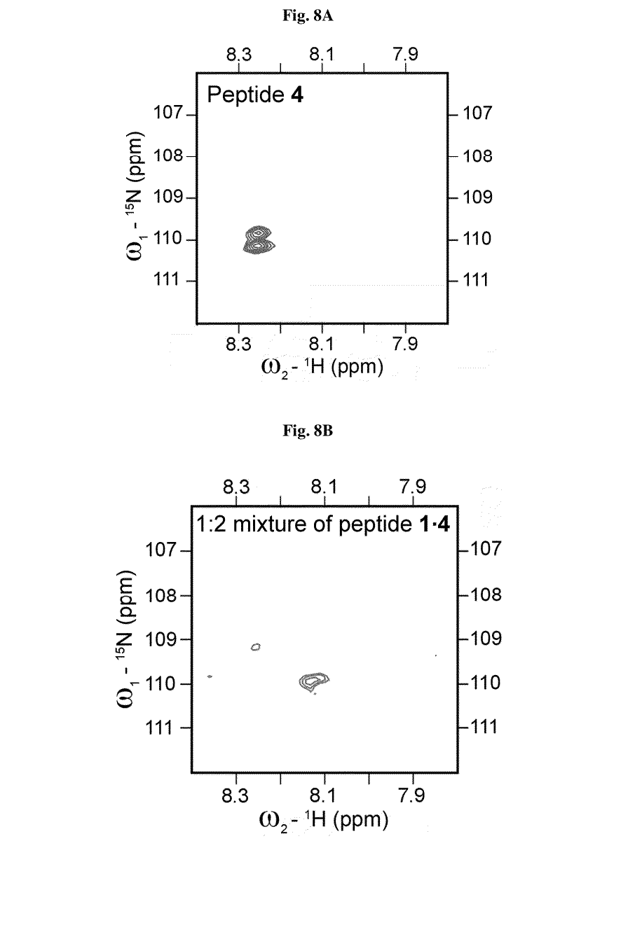

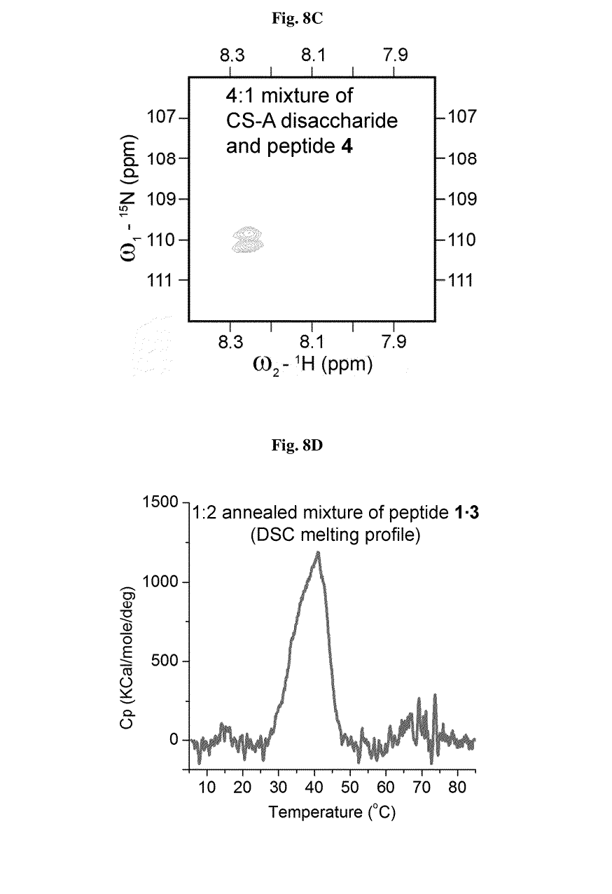

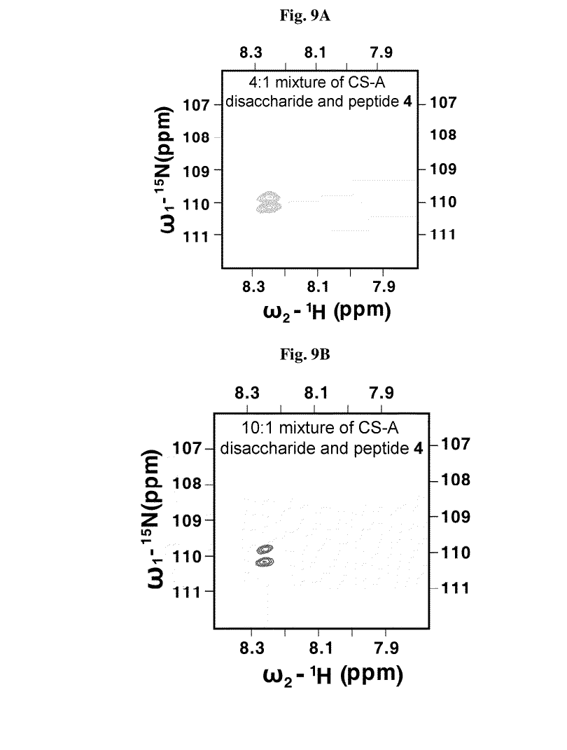

2D solution NMR measurements was therefore performed to further clarify the heterotrimeric formation of 1•3. A glycine residue at the fifth triplet of the WG-(POG)₂-(PRG)₄-(POG)₂ peptide was labeled with ¹⁵N isotope. All samples were annealed followed by incubation at 4° C., and analyzed at 15° C. to avoid monomeric states of peptides in the sample solutions. The ¹H, ¹⁵N-HSQC (Heteronuclear Single-Quantum Correlation) spectrum of the homotrimeric 4 displayed two distinct chemical shifts with similar populations (FIG. 8). This may be attributed to the different chemical environments in CTH as peptide 4 contains two sets of domains, POG and PRG triplets, within the chain. On the other hand, the mixture of 1 and 3 showed one peak corresponding to the main register and additional small cross peaks from minor registers in heterotrimeric assembly. It is noteworthy that no peaks from homotrimeric 4 were observed (FIG. 8). To rule out the possibility of biased results caused by the interactions of pendant CS-A disaccharide with backbone amide, we next examined the influence of pendant sugars on the chemical shifts by adding an excess monovalent CS-A disaccharide to the solution of 4, yet no peak showing a change in chemical shift in HSQC was detected (FIG. 8 and FIG. 9). Finally, differential scanning calorimetry (DSC) was employed to elucidate the thermodynamic nature of heterotrimers 1.3 (FIG. 8). One major endothermic transition was identified in the range of 25-47° C. and Tₘ was determined at around 41.3° C., confirming earlier observations in circular dichroism and HSQC studies, the existence of single major register in CTH of 1•3.

The melting temperatures of the homo- and heterotrimers stated above are presented in Table 1.

TABLE 1
Melting temperatures of homo- and heterotrimers.

| Peptide | Tₘ (° C.)^{[a]} |
|---|---|
| 1 | No triple helix |
| 2 | No triple helix |
| 3 | 39.4 |
| 1•2^{[b]} | 16.9, 17.4^{[c]} |
| 1•3^{[b]} | 42.4, 42.3^{[c]}, 41.3^{[d]} |

^{[a]}The minimum of the derivative of fraction folded plot is used to indicate the melting temperature (Tₘ),
^{[b]}Peptides were mixed together at a ratio of 1:2,
^{[c]}Peptides were preheated to ensure equilibrium,
^{[d]}Tₘ of preheated 1•3 was determined by DSC.

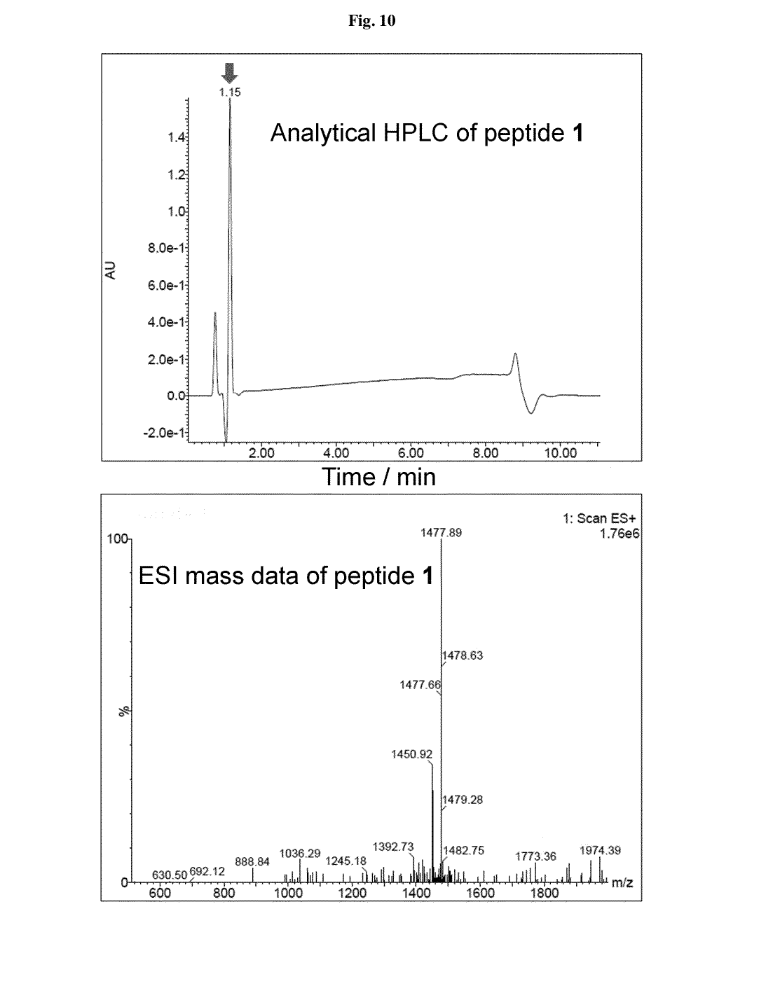

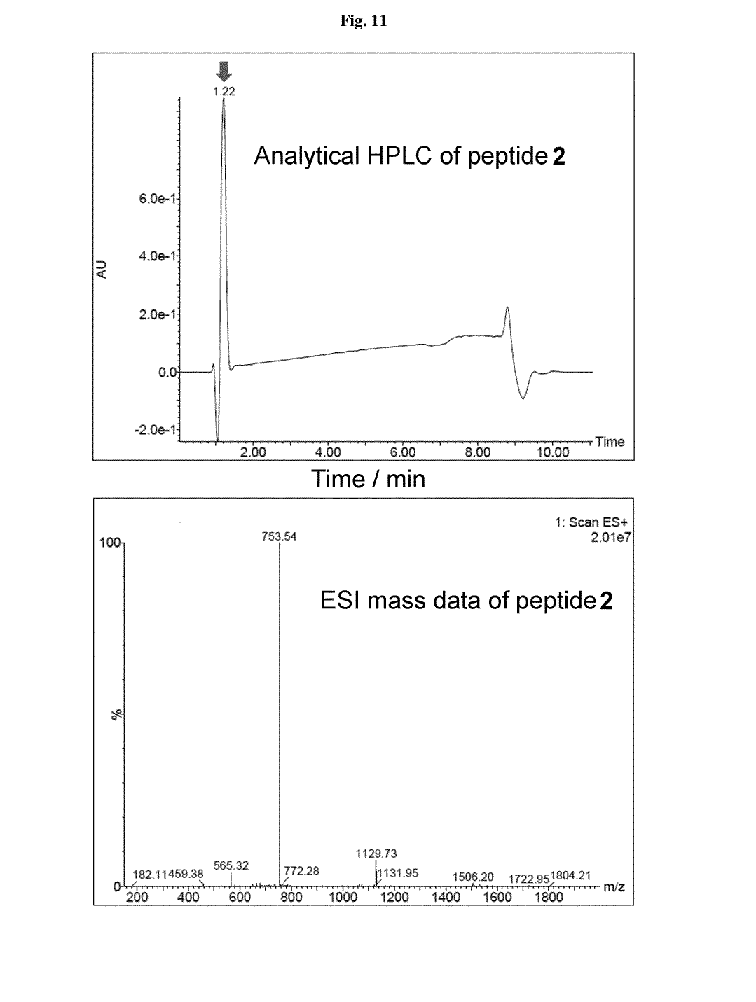

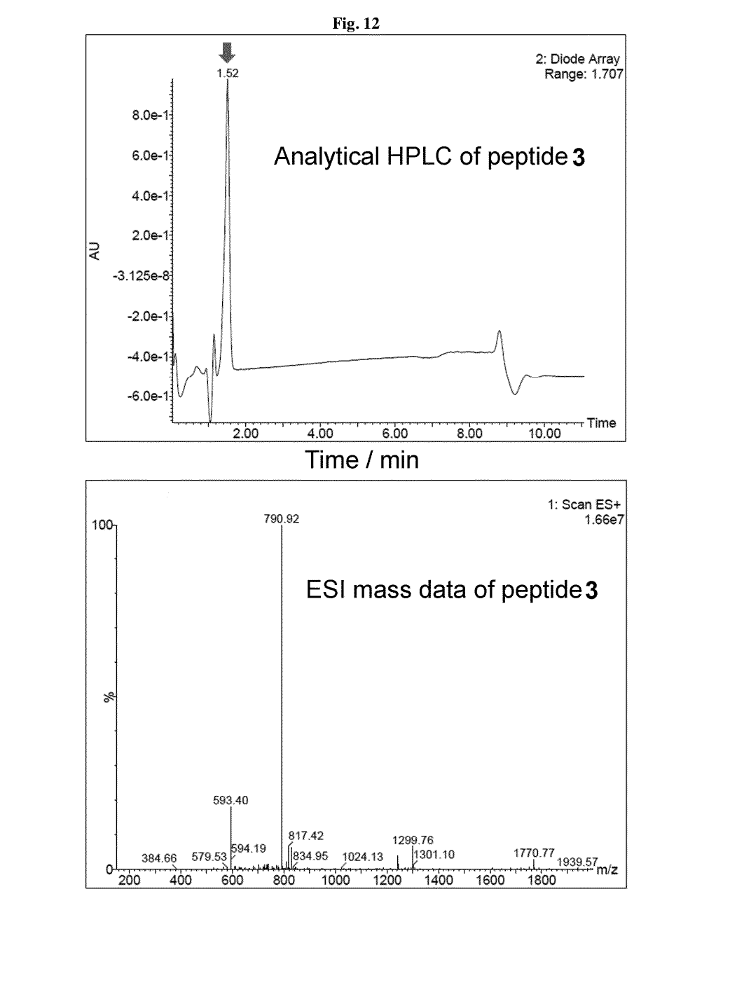

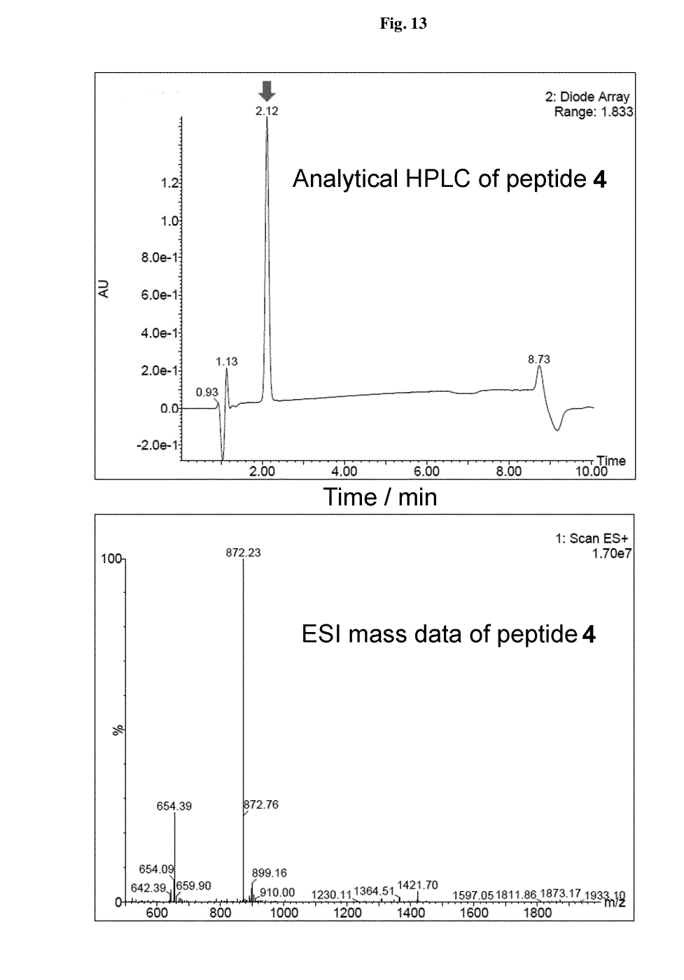

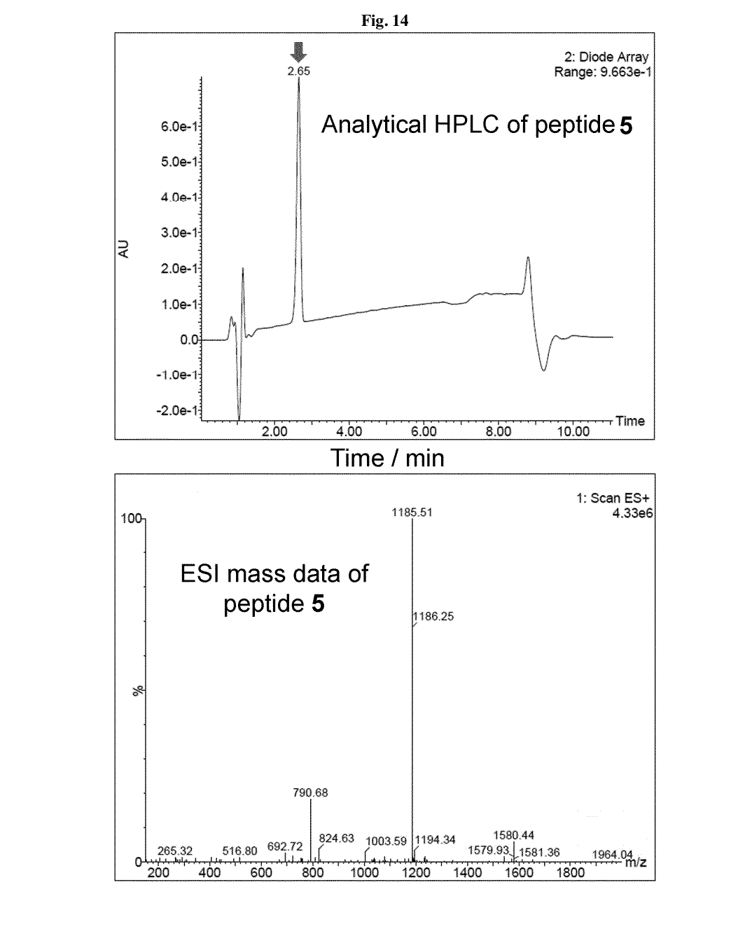

Analytical HPLC traces and ESI mass data for peptides 1 to 5 are shown in FIGS. 10 to 14 respectively.

### EXAMPLE 4

This example describes the procedure for alkyne-functionalised CS-A disaccharide synthesis.

Unless otherwise stated, reactions were performed in flame-dried glassware under argon atmosphere and using anhydrous solvents. All commercially obtained reagents were used as received unless otherwise noted. Thin layer chromatography (TLC) was performed using E. Merck silica gel 60 F254 precoated plates (0.25 mm). Visualization of the developed chromatogram was performed by UV, cerium ammonium molybdate, or ninhydrin stain as necessary. Merck silica gel 60 (particle size 0.040-0.063 mm) was used for flash chromatography. Gel filtration chromatography (Sephadex G-15 ultrafine) was used in order to achieve the purification of peptide 1.

¹H NMR and proton decoupling experiments were recorded on a Bruker AVIII 400 (400 MHz) spectrometer and are reported in parts per million (δ) relative to CDCl₃ (7.26 ppm), CD₃OD (4.87 ppm), D₂O (4.80 ppm), CDCl₃/CD₃OD (1:1) (3.49 ppm), and CDCl₃/CD₂Cl₂ (containing 0.5% TMS)/CD₃OD (1:4.5:4.5) (3.49 ppm).^{[1]} Data for the ¹H NMR spectra are reported as follows: chemical shift (δ ppm), multiplicity (s=singlet, bs=broad singlet, d=doublet, t=triplet, q=quartet, m=multiplet), coupling constant in Hz, and integration. ¹³C NMR spectra were obtained on a Bruker AVIII 400 (100 MHz) spectrometer and are reported in terms of chemical shift. Mass spectra were obtained from Chemical, Molecular and Materials Analysis Centre at the National University of Singapore.

3-Trimethylsilylpropargyl O-(methyl 2,3,4-tri-O-acetyl-β-D-glucopyranosyluronate)-(1→3)-4,6-O-benzylidene-2-deoxy-2-trichloroacetamido-β-D-galactopyranoside 5: Donor 4 (0.500 g, 0.573 mmol) was co-evaporated with toluene (3×5 mL) and dried under vacuum overnight. To a solution of 4 and 3-trimethylsilylpropargyl alcohol (0.425 mL, 2.863 mol) in dry CH₂Cl₂ (7.35 mL) was added 4 Å powdered molecular sieves. The reaction was stirred at room temperature for 30 min, cooled to −78° C., and then stirred for an additional 30 min. Trimethylsilyl trifluoromethanesulfonate (0.227 mM in CH₂Cl₂, 0.115 mmol, 500 μL) at −78° C. was added to the reaction dropwise. The reaction was warmed to −15° C., stirred for 2 hr, and quenched with triethylamine. The reaction mixture was filtered through Celite and concentrated to afford yellow syrup. The product was purified by flash chromatography (1%→2% 2-propanol:CH₂Cl₂) to afford slightly impure 5 as a light yellow solid. The impure 5 was then purified one more time by flash chromatography (2%→5% THF:[1:2 hexanes:CH₂Cl₂]) to afford pure 5 (0.295 g, 61%) as a white solid. R_{f}=0.45 (6:3:1 CH₂Cl₂:hexanes:THF). ¹H NMR (400 MHz, CDCl₃): δ 7.53 (m, 2H, ArH), 7.38-7.33 (m, 3H, ArH), 6.97 (d, J=6.8 Hz, 1H, NH), 5.59 (s, 1H, PhCH), 5.23 (d, J=8.2 Hz, 1H, H-1′), 5.23 (t, J=9.4 Hz, 1H, H-4), 5.16 (t, J=8.8 Hz, 1H, H-3), 5.04 (t, J=7.8 Hz, 1H, H-2), 4.90 (d, J=7.5 Hz, 1H, H-1), 4.77 (dd, J=11.1, 3.4 Hz, 1H, H-3′), 4.46 (d, J=3.3 Hz, 1H, H-4′), 4.40 (s, 2H, CH₂—C≡C), 4.33 (d, J=12.4 Hz, 1H, H-6′), 4.10 (d, J=11.6 Hz, 1H, H-6′), 4.03 (d, J=9.7 Hz, 1H, H-5), 3.83-3.77 (m, 1H, H-2′), 3.72 (s, 3H, OCH₃), 3.55 (s, 1H, H-5′), 2.01 (s, 3H, C(O)CH₃), 2.01 (s, 3H, C(O)CH₃), 2.00 (s, 3H, C(O)CH₃), 1.99 (s, 3H, C(O)CH₃), 0.16 (s, 9H, Si(CH₃)₃); ¹³C NMR (100 MHz, CDCl₃): δ 170.20, 169.57, 169.32, 167.32, 162.27, 137.80, 128.99, 128.24, 126.33, 100.83, 100.40, 100.17, 96.10, 92.54, 92.48, 75.76, 74.22, 72.57, 72.12, 71.40, 69.22, 69.14, 66.76, 56.53, 55.03, 53.04, 20.97, 20.72, 20.63,-0.06; ESI MS: m/z calcd for [C₃₄H₄₂Cl₃NO₁₅Si+Na]⁺: 860.1282, obsd 860.1296.

3-Trimethylsilylpropargyl O-(methyl 2,3,4-tri-O-acetyl-β-D-glucopyranosyluronate)-(1→3)-4,6-O-benzylidene-2-deoxy-2-acetamido-β-D-galactopyranoside 6: Reduction of the trichloroacetamide group was performed using a procedure modified from Belot et. al.^{[2]} Disaccharide 5 (0.353 g, 0.421 mmol) was dissolved in toluene (8.7 mL), and tributylstannane (1132 μL, 4.21 mmol) and 2,2′-azobisisobutyronitrile (34.5 mg, 0.211 mmol) were added. After stirring at rt for 30 min, the reaction mixture was heated to 80° C. and stirred for an additional 4 h 30 min. The reaction was then cooled to rt and concentrated to afford a white solid. The product was purified by flash chromatography (3→11% THF:CH₂Cl₂) to afford the desired acetamide 6 (0.281 g, 91%) as a white solid. R_{f}=0.25 (4% THF:CH₂Cl₂). ¹H NMR (400 MHz, CDCl₃): δ 7.53 (m, 2H, ArH), 7.37-7.32 (m, 3H, ArH), 5.81 (d, J=6.6 Hz, 1H, NH), 5.56 (s, 1H, PhCH), 5.25-5.18 (m, 3H, H-3, H-4, H-1′), J=7.7 Hz, 1H, H-2), 4.92-4.88 (m, 2H, H-1, H-3′), 4.43-4.36 (m, 3H, H-4′, CH₂—C≡), 4.30 (d, J=12.5 Hz, 1H, H-6′), 4.08-4.02 (m, 2H, H-5, H-6′), 3.70 (s, 3H, OCH₃), 3.53 (s, 1H, H-5′), 3.47-3.41 (m, 1H, H-2′), 2.02 (s, 6H, NHC(O)CH₃, C(O)CH₃), 2.00 (s, 3H, C(O)CH3), 1.98 (s, 3H, C(O)CH₃), 0.18 (s, 9H, Si(CH₃)₃); ¹³C NMR (100 MHz, CDCl₃): δ 171.31, 170.29, 169.59, 169.18, 167.47, 137.90, 128.92, 126.37, 100.85, 100.81, 100.57, 97.07, 91.88, 75.98, 75.16, 72.52, 72.03, 71.74, 69.29, 69.18, 66.60, 56.75, 54.31, 52.98, 23.98, 20.86, 20.75, 20.65, −0.07; ESI MS: m/z calcd for [C₃₄H₄₅NO₁₅Si+Na]⁺: 758.2456, obsd 758.2469.

2-Propargyl O-(methyl 2,3,4-tri-O-acetyl-β-D-glucopyranosyluronate)-(1→3)-2-deoxy-2-acetamido-β-D-galactopyranoside 7: The acetamide 6 (0.353 g, 0.480 mmol) was dissolved in AcOH/water (4:1, 3.0 mL) and stirred at 80° C. After 30 min, the reaction mixture was cooled and concentrated. The resulting concentrate was co-evaporated with toluene (3×3 mL) for the complete removal of AcOH.

To a solution of crude diol (0.242 g, 0.374 mmol) in THF (3.7 mL) was added TBAF (1 M solution in THF, 0.448 mmol, 448 μL) and the mixture stirred at 0° C. for 1.5 hr. At this time the addition of Amberlyst IR-120 resin was made and the reaction stirred for a further 30 min. After filtration, the mixture was concentrated to afford a light yellow solid. The residue was purified via flash chromatography (5 7% MeOH:CH₂Cl₂) to afford the desired compound (0.147 g, 53%) as a white solid. R_{f}=0.30 (10% MeOH:CH₂Cl₂). ¹H NMR (400 MHz, CD₃OD): δ 5.35 (t, J=6.6 Hz, 1H, H-3), 5.11 (t, J=9.8 Hz, 1H, H-4), 5.00 (dd, J=8.0 Hz, 9.4 Hz, 1H, H-2), 4.91 (d, J=8.0 Hz, 1H, H-1), 4.66 (d, J=8.2 Hz, 1H, H-1′), 4.43-4.37 (m, 2H, CH₂—C≡C), 4.29 (d, J=10.0 Hz, 1H, H-5), 4.07 (bs, 1H, H-4′), 4.00-3.98 (m, 1H, H-2′), 3.91-3.89 (m, 1H, H-3′), 3.81-3.72 (m, 2H, H-6′), 3.75 (s, 3H, OCH₃), 3.54 (t, J=5.8 Hz, 1H, H-5′), 2.87 (t, J=2.32 Hz, 1H, C≡CH), 2.06 (s, 3H, NHC(O)CH₃), 2.03 (s, 3H, C(O)CH₃), 2.01 (s, 3H, C(O)CH₃), 2.00 (s, 3H, C(O)CH₃); ¹³C NMR (100 MHz, CD₃OD): δ 173.41, 171.48, 171.21, 169.31, 102.60, 100.50, 81.36, 80.14, 76.47, 76.13, 73.45, 72.95, 72.57, 70.84, 69.17, 62.40, 56.43, 53.41, 52.56, 23.40, 20.97, 20.77, 20.49, 20.41; ESI MS: m/z calcd for [C₂₄H₃₃NO₁₅+Na]⁺: 598.1748, obsd 598.1760.

2-Propargyl O-(methyl 2,3,4-tri-O-acetyl-β-D-glucopyranosyluronate)-(1→3)-6-O-benzoyl-2-deoxy-2-acetamido-p-D-galactopyranoside 8; Disaccharide 7 (30 mg, 0.052 mmol) was dissolved in anhydrous pyridine (2.1 mL) and this was added benzoyl cyanide (20.6 mg, 0.157 mmol) and 4-(dimethylamino)pyridine (10.2 mg, 0.084 mmol). The reaction was stirred at rt for 3 hr and then concentrated to afford a yellow solid. The residue was purified via flash chromatography (1→4% MeOH:CH₂Cl₂) to afford the desired compound 8 (27.8 mg, 78%) as a white solid. R_{f}=0.45 (10% MeOH:CH₂Cl₂). ¹H NMR (400 MHz, CDCl₃/CD₃OD (1:1)): δ 8.19-8.17 (m, 2H, ArH), 7.76-7.70 (m, 1H, ArH), 7.63-7.59 (m, 2H, ArH), 5.45 (t, J=9.5 Hz, 1H, H-3), 5.27 (t, J=9.6 Hz, 1H, H-4), 5.00 (t, J=9.4 Hz, 1H, H-2), 4.98 (d, J=8.5 Hz, 1H, H-1′), 4.97 (d, J=8.0 Hz, 1H, H-1), 4.72-4.65 (m, 2H, H-6′, H-6′), 4.52-4.45 (m, 2H, CH₂—C≡C), 4.35-4.30 (m, 3H, H-5, H-3′, H-4′), 4.05 (t, J=6.2 Hz, 1H, H-5′), 4.01-3.90 (m, 1H, H-2′), 3.83 (s, 3H, OCH₃), 2.32 (t, J=2.32 Hz, 1H, C≡CH), 2.21 (s, 3H, NHC(O)CH₃), 2.19 (s, 3H, C(O)CH₃), 2.17 (s, 3H, C(O)CH₃), 2.14 (s, 3 H; C(O)CH₃); ¹³C NMR (100 MHz, CD₃OD): δ 174.08, 172.31, 171.99, 171.95, 169.86, 168.62, 135.18, 131.66, 131.48, 130.36, 103.24, 100.26, 81.16, 80.53, 76.91, 74.14, 73.91, 73.57, 73.08, 71.37, 69.75, 65.43, 57.45, 54.71, 53.79, 24.62, 22.07, 22.00; ESI MS: m/z calcd for [C₃₁H₃₇NO₁₆+Na]⁺: 702.2005, obsd 702.2027.

2-Propargyl O-(sodium-β-D-glucopyranosyluronate)-(1→3)-4-O-sodium sulfonato-2-deoxy-2-acetamido-β-D-galactopyranoside 9: To a solution of compound 8 (0.020 g, 0.029 mmol) in anhydrous DMF (1.5 mL) was added sulfur trioxide trimethylamine complex (SO₃.TMA) (0.025 g, 0.180 mmol). The reaction mixture was stirred at 50° C. for 36 hr and then cooled to rt. The product was purified on Sephadex LH-20 (50% MeOH:CH₂Cl₂), followed by silica gel chromatography (5%→10% MeOH:CH₂Cl₂) to afford a sulfated disaccharide (0.016 g, 73%) as a white solid. R_{f}=0.18 (10% MeOH:CH₂Cl₂). ¹H NMR (400 MHz, 1:4.5:4.5 CDCl₃:CD₃OD:CD₂Cl₂ containing 0.05% v/v TMS): δ 8.16-8.12 (m, 2H, ArH), 7.69-7.65 (m, 1H, ArH), 7.56-7.52 (m, 2H, ArH), 5.40 (t, J=9.6 Hz, 1H, H-3), 5.27 (t, J=9.8 Hz, 1H, H-4), 5.21 (t, J=9.5 Hz, 1H, H-2), 4.98 (d, J=1.9 Hz, 1H, H-4′), 4.91 (d, J=8.1 Hz, 1H, H-1′), 4.89 (d, J=8.8 Hz, 1H, H-1), 4.79-4.69 (m, 2H, H-6′, H-6′), 4.42-4.40 (m, 2H, CH₂—C≡C), 4.30-4.21 (m, 2H, H-5, H-3′), 4.10-4.05 (m, 1H, H-5′), 3.94-3.86 (m, 1H, H-2′), 3.81 (s, 3H, OCH₃), 2.63 (t, J=2.32 Hz, 1H, C≡CH), 2.17 (s, 3H, NHC(O)CH₃), 2.12 (s, 3H, C(O)CH₃), 2.09 (s, 3H, C(O)CH₃), 2.08 (s, 3H, C(O)CH₃); ¹³C NMR (100 MHz, CD₃OD): δ 174.34, 172.73, 172.14, 172.04, 170.77, 168.57, 135.01, 131.83, 131.48, 130.27, 102.79, 99.91, 80.39, 79.57, 78.96, 76.83, 74.07, 73.72, 73.68, 72.92, 71.29, 66.02, 57.33, 55.61, 55.53, 55.34, 55.07, 54.98, 53.92, 51.17, 50.95, 50.74, 50.53, 24.56, 22.25, 22.07, 22.01; ESI MS: m/z calcd for [C₃₁H₃₇NO₁₉S+Na]⁺: 782.1573, obsd 782.1588.

The sulfated compound (160 mg, 0.211 mmol) was dissolved in THF (684 μL) and H₂O (338 μL) and cooled to 0° C. To this were added 1 M aq. LiOH (270 μL) and 30% H₂O₂ (135 μL). The reaction stirred at 0° C. for 1 hr and at rt for 12 hr. At this time, 4 M aq. NaOH (203 μL) and MeOH (1008 μL) were added and the reaction stirred for another 12 hr. It was then neutralized with Amberlyst IR-120 resin, filtered, and lyophilized to afford an orange solid. The product was purified by Sephadex G-15 (100% H₂O) and lyophilized to afford 9 as a white solid (103 mg, 87%). ¹H NMR (400 MHz, D₂O): δ 4.86 (d, J=2.7 Hz, 1H, H-4′), 4.77 (d, J=8.1 Hz, 1H, H-1′), 4.51 (d, J=7.7 Hz, 1H, H-1), 4.45 (t, J=2.1 Hz, 2H, CH₂—C≡C), 4.14-4.04 (m, 2H, H-2′, H-3′), 3.87-3.79 (m, 3H, H-5′, H-6′, H-6′), 3.74 (d, J=9.7 Hz, 1H, H-5), 3.57 (t, J=9.0 Hz, 1H, H-4), 3.49 (t, J=9.2 Hz, 1H, H-3), 3.38 (dd, J=9.2, J=7.8 Hz, 1H, H-2), 2.93 (t, J=2.4 Hz, 1H, 2.05 (s, 3H, NHC(O)CH₃); ¹³C NMR (100 MHz, D₂O): δ 175.22, 174.90, 103.30, 99.37, 78.69, 76.30, 76.21, 76.01, 75.03, 74.88, 74.60, 72.45, 71.65, 60.95, 56.70, 51.53. 22.27; ESI MS: m/z calcd for [C₁₇H₂₅NO₁₅S—H]⁻: 514.0867, obsd 514.0867.

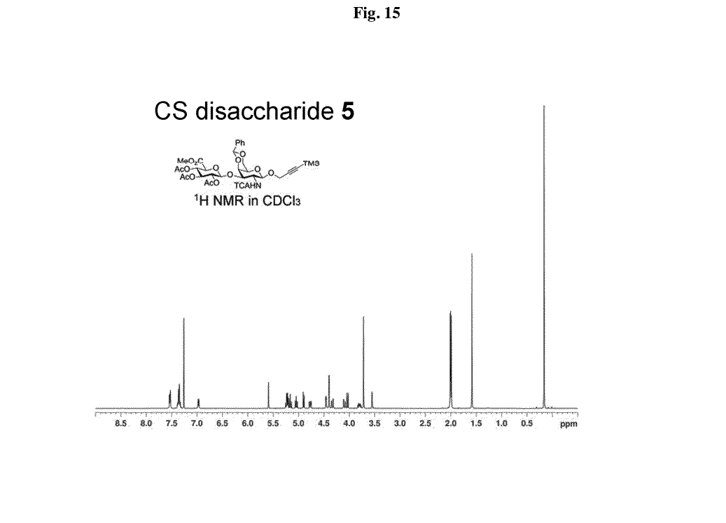

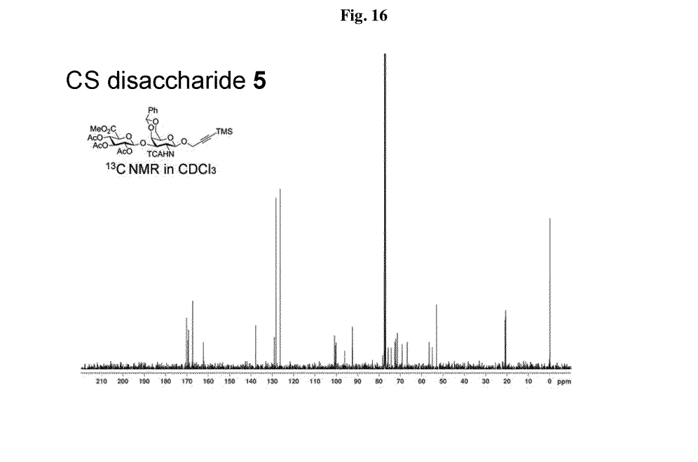

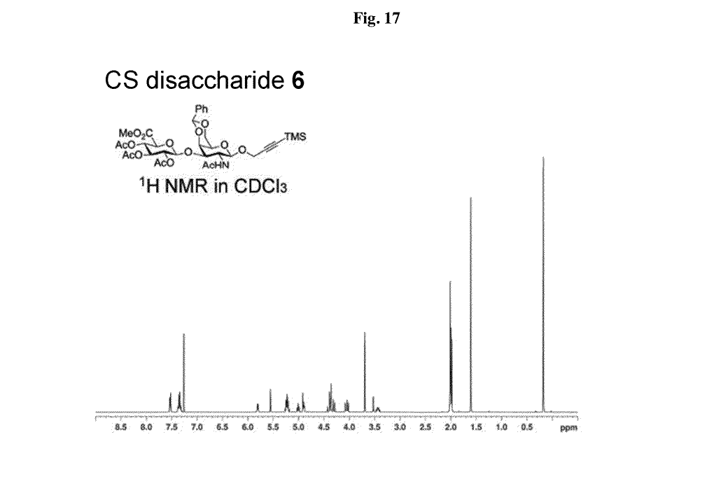

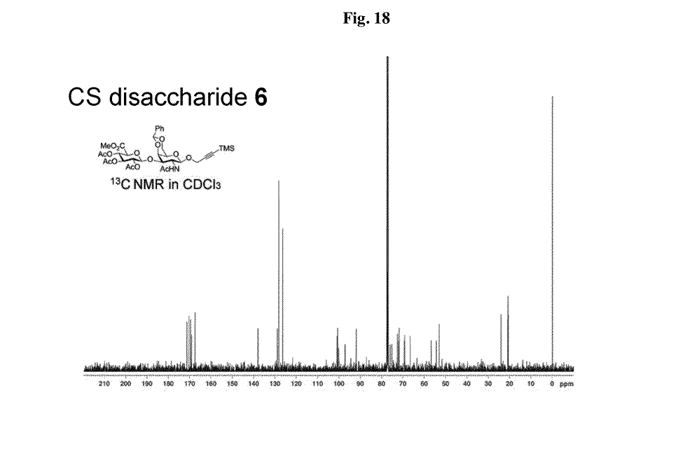

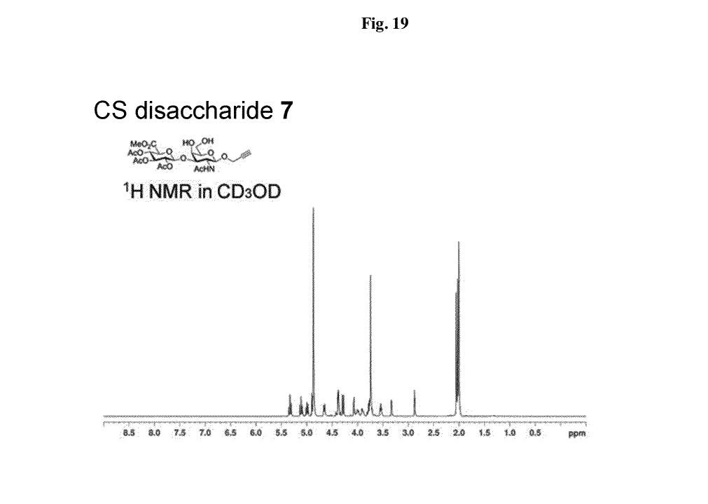

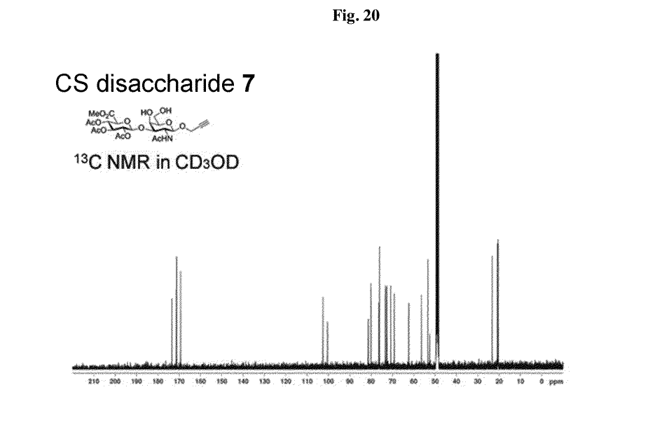

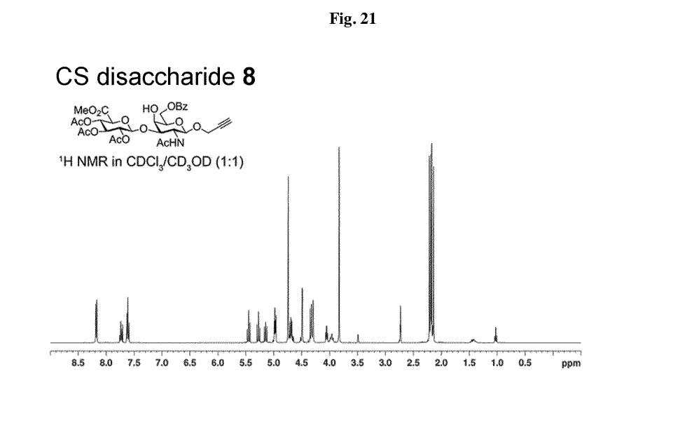

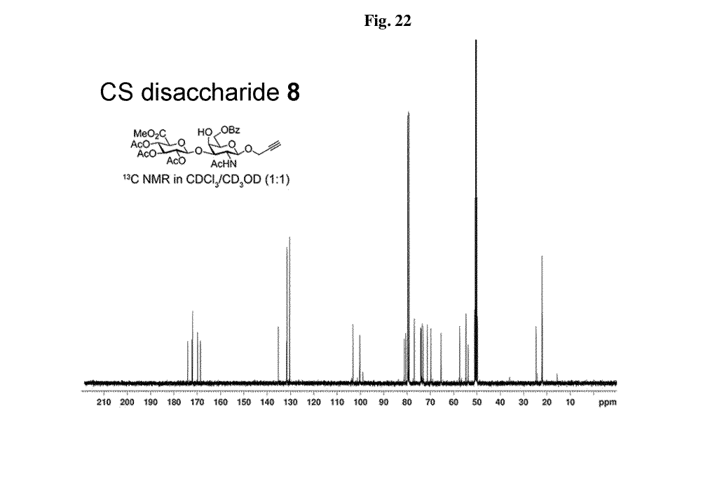

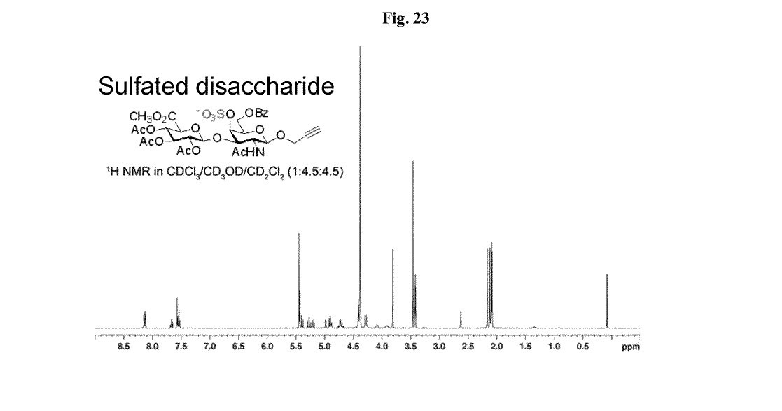

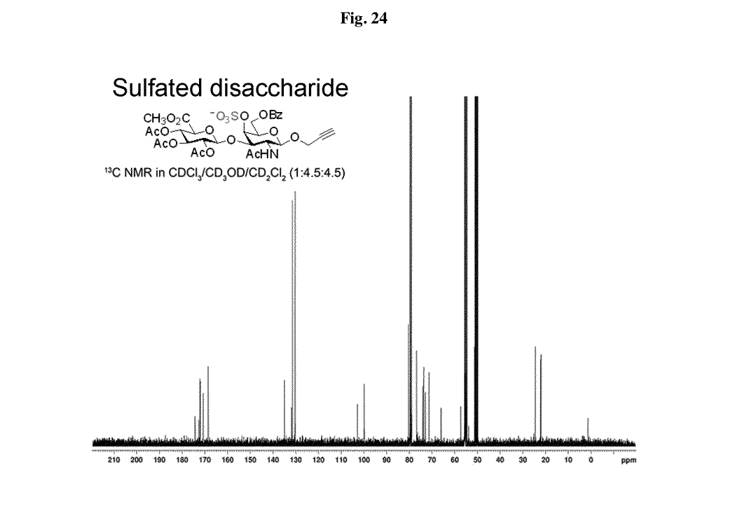

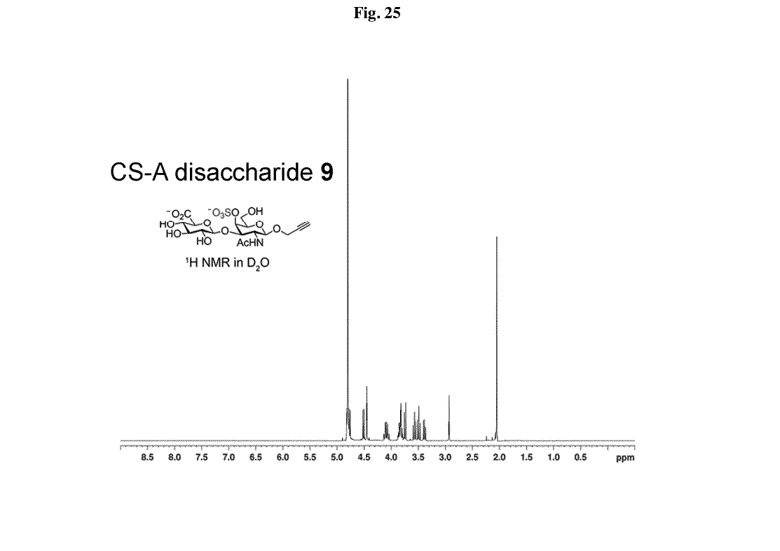

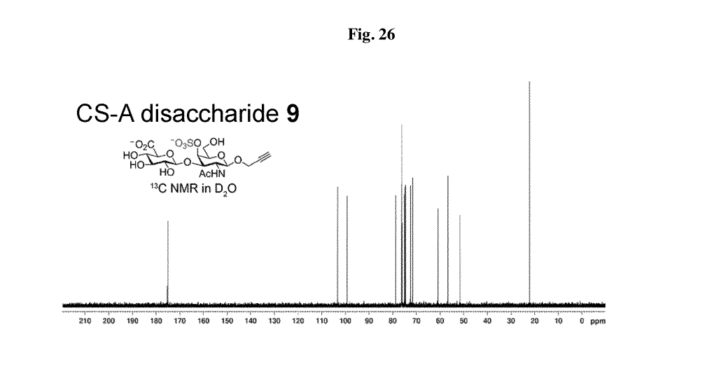

The ¹H NMR and ¹³C NMR spectra for compounds 5, 6, 7, 8 and 9 are shown in FIGS. 15 to 26.

### EXAMPLE 5

This example describes the general procedure for solid phase peptide synthesis.

Synthesis of the peptide library was performed by using an automatic synthesizer Titan 357 (AAPPTEC). 50 mg of ChemMatrix® resins (0.48 mmol/g) were swelled in NMP (1 ml) for 5 min in a Reaction Vessel (RV). With the liquid drained, 20% piperidine in NMP (1 ml, v/v) was added and the RV was vortexed for 3 min. The liquid was drained and a fresh solution of 20% piperidine in NMP (1 ml, v/v) was added and the RV was vortexed for another 12 min. The resulting beads were thoroughly washed by NMP (1 ml×2), methanol (1 ml×2), and DCM (1 ml×2). With the resulting resins swelled with NMP (1 ml) for 15 min, Fmoc-protected amino acid (2.5 equiv, 0.2 M solution in NMP) was added to the RV, as well as TBTU (2.5 equiv, 0.2 M solution in NMP) and DIEA (5.0 equiv, 0.5 M in NMP). The resulting mixture was vortexed for 45 min. With the liquid drained, the resulting beads were thoroughly washed by NMP (1 ml×3). The coupling step was repeated until the desired sequence of peptide attained. N-terminus was modified with acetyl group by treatment of acetic anhydride (10 equiv, 0.5 M solution) and DIEA (20 equiv, 0.5 M in NMP) for 10 min. The resins were washed by NMP (1 ml×3) and transferred in a 4 ml reactor equipped with a filter, using DCM (2 ml×3). After the resins were dried under reduced pressure for 2 h, the peptide was cleaved in a cleavage cocktail of TFA-water-TIS, (1.5 ml, 94/3/3, v/v) for 2 h on a 180-degree shaker, while all the acid-labile protective groups in the residues' were also detached. The solution was collected and concentrated in a continuous flow of nitrogen and the crude peptides were precipitated in diethylether. The resulting white solid was then purified to >98% in purity by a preparative HPLC (Gilson) on a C-18 reversed phase preparative column (Kromasil®, 21.2 mm×250 mm) using water/acetonitrile containing 0.1% trifluoroacetic acid as the mobile phase and characterized with Waters Acquity UltraPerformance LC and mass spectrometry (UPLC/MS).

HPLC conditions and ESI mass data for peptides 1 to 5 are provided in Tables 2 and 3 respectively.

TABLE 2
Analytical HPLC conditions for peptides.

| Peptide | HPLC condition * | Retention time |
|---|---|---|
| Peptide 1 | 5-60% A over 8 min | 1.15 min |
| Peptide 2 | 5-60% A over 8 min | 1.22 min |
| Peptide 3 | 5-60% A over 8 min | 1.52 min |
| Peptide 4 | 5-60% A over 8 min | 2.12 min |
| Peptide 5 | 5-60% A over 8 min | 2.65 min |

* A: CH₃CN/0.1% TFA, B: H₂O/0.1% TFA.

TABLE 3
ESI-MS Data of peptides.

| Peptide | MS, calculated | ESI-MS, observed |
|---|---|---|
| Peptide 1 | 4428.46 | 1477.89 ([M + 3H]³⁺), |
| | | 1450.92 ([M − SO₃ + 3H]³⁺), |
| Peptide 2 | 2256.20 | 1129.73 ([M + 2H]²⁺), |
| | | 753.54 ([M + 3H]³⁺), |
| | | 565.32 ([M + 4H]⁴⁺) |
| Peptide 3 | 2368.23 | 1299.76 ([M + 2TFA]²⁺), |
| | | 790.92 ([M + 3H]³⁺), |
| | | 593.40 ([M + 4H]⁴⁺) |
| Peptide 4 | 2612.33 | 1421.70 ([M + 2TFA]²⁺), |
| | | 872.23 ([M + 3H]³⁺), |
| | | 654.39 ([M + 4H]⁴⁺) |
| Peptide 5 | 2368.08 | 1185.51 ([M + 2H]²⁺), |
| | | 790.68 ([M + 3H]³⁺) |

### EXAMPLE 6

This example describes the procedure for the synthesis of peptide 1 via click reaction.

A small vial was charged with alkyne-functionalized CS-A disaccharide (4.6 mg, 4.4 equiv., 8.559 mol), peptide 5 (4.6 mg, 1.0 equiv., 1.941 μmol), Tris[(1-benzyl-1H-1,2,3-triazol-4-yl)methyl]amine (TBTA, 0.3 equiv. per azide) and a small stir bar under argon atmosphere. The mixture was dissolved in degassed DMSO (150 μL), and the desired amount of copper (I) iodide stock solution (3 μL of 7.876 mM stock solution in DMSO, 0.3 mol % per azide), and DIPEA (18 53 equiv., 0.103 mmol) were added. The reaction mixture was stirred at room temperature for 5 days. The consumption of peptide 5 was monitored by analytical reverse phase HPLC equipped with C-18 column. After the completion, solvent was removed and the white precipitate was dissolved in 200 μL of aqueous 6 M NaCl and purified by Sephadex G-15 column (100% H₂O) to afford peptide 1 as a white solid upon lyophilization.

### EXAMPLE 7

This example describes circular dichroism (CD) analysis.

CD spectra were recorded on an Aviv 410 circular dichroism spectrometer equipped with temperature controller. Peptide solutions at concentrations of 200 μM were used. All sample solutions made in 10 mM sodium phosphate-dibasic buffer (pH 7.0) were equilibrated for 24 hr at 4° C. before CD measurements. A Quartz Cell of 1 mm path length was used. For wavelength scan, temperature was increased to the desired points at a rate of 10° C./hr and the sample solutions were equilibrated for 10 min before the measurements. Spectra were recorded from 260 to 190 nm and mean residue ellipticity [θ] was calculated as follows;

[θ]=θ/(10·N·c·l)

θ represents the ellipticity in millidegrees, N the number of amino acid residues, c the molar concentration in mol·L⁻¹, and l the cell path length in cm. Thermal denaturation curves were obtained at final peptide concentrations of 0.2 mM and molar ellipticity at 225 nm was measured as a function of temperature in a range of 5 to 80° C. at a heating rate of 10° C./hr. All annealed samples were heated for 30 min at 85° C. and then cooled down to 4° C. at a rate of 1° C./min followed by equilibration for 24 hr at 4° C. before the measurements. The melting temperatures were determined from the minimum points in the first derivative of the melting curves.

### EXAMPLE 8

This example describes ¹H, ¹⁵N-Heteronuclear Single Quantum Coherence (HSQC) experiments.

¹H, ¹⁵N-HSQC experiments were recorded on a Bruker Avance 800 spectrometer equipped with a temperature unit and CryoProbe. All sample solutions were prepared at 200 μM of total peptide concentration in 10 mM sodium phosphate-dibasic buffer (pH 7.0) containing 5% D₂O. Both homotrimeric and heterotrimeric samples were heated for 30 min at 85° C. and then cooled down to 4° C. at a rate of 1° C./min followed by equilibration for 24 hr at 4° C. before the measurements. The measurements were performed at 15° C. to avoid any monomeric states of peptides in the sample solutions. A total of 1280×128 complex points in 4 scans were acquired for the ¹H, ¹⁵N-HSQC experiments using a spectral window of 11160 Hz in the hydrogen dimension and 1621 Hz in the nitrogen dimension. The spectra were processed using NMRpipe and analyzed using Sparky. Square Cosine bell window functions were used as apodization functions and the data was zero-filled to the next power of two in both dimensions. Baseline corrections were applied when necessary.

### EXAMPLE 9

This example describes differential scanning calorimetry (DSE) experiments.

DSC experiments were performed on a MicroCal VP-DSC System from GE Healthcare Life Sciences. Peptide mixture 1•3 and buffer concentrations were the same as in the CD experiments: 200 μM total peptide concentration and 10 mM sodium phosphate-dibasic buffer (pH 7.0). Peptide mixture 1.3 was heated for 30 min at 85° C. and then cooled down to 4° C. at a rate of 1° C./min followed by equilibration for 24 hr at 4° C. before the measurements. The measurements were performed in the temperature range of 5 to 80° C. at a heating rate of 10° C./hr. Peptide curve was baseline subtracted based on curve of the buffer prior to data analysis. Data analysis was performed with MicroCal-enabled Origin software.

Given their physiological significance in vivo, collagen-GAG composites have been an important part of a growing arsenal of biomimetic scaffolds to replace tissues. Nevertheless, the drawbacks associated with natural materials have hindered efforts to develop more sophisticated biomaterials. As demonstrated herein, GAG-containing collagen peptides on the basis of complementary electrostatic interactions have been successfully engineered. It has also been demonstrated that negatively charged chondroitin sulfate motifs can be efficiently introduced into CTH, thereby generating the high quality of heterotrimers. This design represents the hybridization of two different biocomponents in a single framework. This approach will greatly simplify the construction of complex GAG-collagen-based scaffolds, providing synthetically accessible, biologically programmable mimetic structures.

1. A collagen molecule comprising three chains wherein at least one chain comprises at least one repeating unit of the general formula:
(X1-X2-G)n;
wherein X1 or X2 can be any amino acid or amino acid derivative provided that at least one of X1 or X2 is a glycosaminoglycan moiety;
G is glycine; and
n is a positive integer and is at least 1.

2. The collagen molecule according to claim 1, wherein n is at least 2, or at least 5, or at least 10, or at least 20, or at least 50, or at least 100, or at least 200, or at least 500, or at least 1000.

3. The collagen molecule according to claim 1, wherein the glycosaminoglycan moiety is comprised of at least one disaccharide unit.

4. The collagen molecule according to claim 3, wherein the glycosaminoglycan moiety contains at least one negatively charged group.

5. The collagen molecule according to claim 4, wherein the negatively charged group is sulfate or carboxylate.

6. The collagen molecule according to claim 1, wherein the glycosaminoglycan is selected from the group consisting of: a chondroitin sulfate, a heparin, a heparin sulfate, a dermatan sulfate, a hyaluronan, a keratan sulfate and unsulfated chondroitin.

7. The collagen molecule according to claim 6, wherein the chondroitin sulfate is selected from the group consisting of: chondroitin sulfate A, chondroitin sulfate C, chondroitin sulfate D, chondroitin sulfate E, chondroitin sulfate U, chondroitin sulfate K and chondroitin sulfate L.

8. The collagen molecule according to claim 7, wherein the chondroitin sulfate is chondroitin sulfate A or chondroitin sulfate U.

9. The collagen molecule according to claim 1, wherein the amino acid is a positively charged amino acid.

10. The collagen molecule according to claim 9, wherein the positively charged amino acid is selected from the group consisting of: Asp, Glu, Lys, Arg and His.

11. The collagen molecule according to claim 1, wherein at least one of the collagen chains has a general formula selected from the group consisting of:
Ac-(PXG)₃-(CS-U-XG)₂-(PXG)₃-C(O)NH₂,
Ac-(PXG)₃-(CS-A-XG)₂-(PXG)₃-C(O)NH₂,
Ac-(PXG)₂-(CS-A-XG)₄-(PXG)₂-C(O)NH₂,
wherein Ac is acetyl;
P is proline;
G is glycine;
CS-U is unsulfated chondroitin sulfate;
CS-A is chondroitin sulfate A; and
X is hydroxyproline.

12. The collagen molecule according to claim 11, wherein (CS-A-XG) has the following formula:

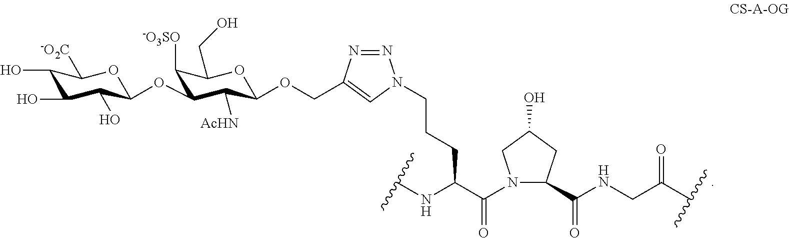

13. The collagen molecule according to claim 11, wherein (PXG) has the following formula:

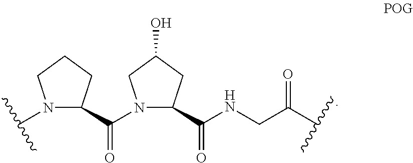 [SMILES: *C(=O)CNC(=O)[C@@H]1C[C@@H](O)CN1C(=O)[C@@H]1CCCN1*]

14. (canceled)

15. A method of treating a patient in need of therapy, comprising administering a collagen molecule according to claim 1.

16. A cosmetic product or scaffold or glycosaminoglycan-based pharmaceutical comprising a collagen molecule according to claim 1.

17. A collagen molecule according to claim 1 for use in any one of tissue engineering, skin tissue engineering, wound healing or drug delivery.

18.-23. (canceled)
